SEQUENCE LISTING (5 sequences)

SEQ ID NO: 1 - protein, 15 aa, synthetic construct
EAISPPDAASAAPLR

SEQ ID NO: 2 - protein, 18 aa, synthetic construct
LGNTISSLFGGGTTPDAK

SEQ ID NO: 3 - protein, 24 aa, synthetic construct
EAISPPDAASAAPLRTITADTFRK

SEQ ID NO: 4 - protein, 15 aa, synthetic construct
SAAPLRTITADTFRK

SEQ ID NO: 5 - RNA, 20 nt, synthetic construct
  source 1..20: mol_type = other RNA
tttttgaagggtgtgatgct

(19) United States
(12) Patent Application Publication
(10) Pub. No.: US 2026/0251636 A1
(43) Pub. Date: Aug. 27, 2026

(54) METHOD FOR IDENTIFYING TUMOR-SPECIFIC CELL SURFACE O-GL YCOPEPTIDES

(71) Applicants: VIB VZM, Gent (BE); UNIVERSITEIT GENT, Gent (BE)

(72) Inventors: Nico Callewaert, Deinze (BE); Wannes Weyts, Gent (BE); Simon Devos, Gent (BE); An Staes, Gent (BE); Hans Demol, Gent (BE)

(21) Appl. No.: 19/163,184

(22) PCT Filed: Mar. 6, 2024
(86) PCT No.: PCT/EP2024/055880

§ 371 Date: Sep. 8, 2025

(30) Foreign Application Priority Data
Mar. 6, 2023 (EP) 23160180.8

(51) Int. Cl. G01N 33/53

(52) U.S. Cl. CPC G01N 33/5308; G01N 2400/00

(57) ABSTRACT

A field of glycoproteomics for identifying novel cell surface O-linked glycopeptide epitopes. The disclosure relates to methods for identifying O-linked glycopeptides, more specifically, Tn- or SiaTn-antigen O-glycosylation sites on cell surface proteins, the method comprising a combined workflow of enzymatic treatments of cells or tissue samples to allow specific enrichment and labelling of Tn antigens, followed by mapping of the O-glycopeptides through LC-MS/MS. More specifically, the disclosure relates to a chemo-enzymatic method to produce labelled O-glycan-peptides from a plasmamembrane-protein extract sample, wherein Sialyltransferase (ST3Gal1/ST3Gal3) treatment is used to protect free galactose against a follow-on treatment with Galactose oxidase, wherein the oxidized glycans are simultaneously labelled. The disclosure further relates to a tandem mass spectrometry method using an enriched TMT-labelled (Sia)Tn-antigen glycopeptides in a dual fragmentation triggered approach to resolve the identity, localization and quantification of the O-linked glycan peptides.

### CROSS-REFERENCE TO RELATED APPLICATIONS

This application is a national phase entry under 35 U.S.C. § 371 of International Patent Application PCT/EP2024/055880, filed Mar. 6, 2024, designating the United States of America and published as International Patent Publication WO 2024/184407 A1 on Sep. 12, 2024, which claims the benefit under Article 8 of the Patent Cooperation Treaty of European Patent Convention patent application Ser. No. 23/160,180.8, filed Mar. 6, 2023.

### STATEMENT REGARDING ELECTRONIC FILING OF A SEQUENCE LISTING

The XML file named “6ZZ9873-801_PCT.xml (Attach_4) From FA” created on Sep. 7, 2025, with a size of 5,432 bytes is hereby incorporated herein in its entirety by this reference.

### TECHNICAL FIELD

This disclosure relates to the field of glycoproteomics for identifying novel cell surface O-linked glycopeptide epitopes. The disclosure relates to methods for identifying O-linked glycopeptides, more specifically, Tn- or SiaTn-antigen O-glycosylation sites on cell surface proteins, the method comprising a combined workflow of enzymatic treatments of cells or tissue samples to allow specific enrichment and labelling of Tn antigens, followed by mapping of the O-glycopeptides through LC-MS/MS. More specifically, the disclosure relates to a chemo-enzymatic method to produce labelled O-glycan-peptides from a plasmamembrane-protein extract sample, wherein Sialyltransferase (ST3Gal1/ST3Gal3) treatment is used to protect free galactose against a follow-on treatment with Galactose oxidase, wherein the oxidized glycans are simultaneously labelled. The disclosure further relates to a tandem mass spectrometry method using an enriched TMT-labelled (Sia)Tn-antigen glycopeptides in a dual fragmentation triggered approach to resolve the identity, localization and quantification of the O-linked glycan peptides.

### BACKGROUND

Almost all cancer cells undergo genetic changes that influences the O-Glycosylation pathway, leading to both the Tn-antigen and SiaTn-antigen being very prevalent on almost all cancer cells, already in early onset and remaining stable during progression. Tn antigen (Tn), which is an N-acetylgalactosamine (GalNAc) attached to protein Ser/Thr residues via an O-linked glycosidic linkage, or alternatively, SiaTn antigen, or STn, with in addition sialic acid monosaccharide. Tn is expressed in 10-90% of solid tumors but rarely expressed in healthy adults. Tn-specific glycosylation sites are thus known for their use in early diagnostics and prognostics of cancers. Moreover, both antigens are very close to the peptide backbone, making it possible to develop highly selective antibodies that recognize both the peptide backbone and the sugar with one paratope, thereby providing a therapeutic utility. In immunotherapy, specific epitopes are available in leukemias, though in (adeno) carcinomas, there is a an issue with selectivity for targets, so there is a need to discover novel highly specific epitopes as target for novel immunotherapy.

Although Tn is structurally simple, identification of its glycosylation sites and the carrier proteins in the complex samples is highly challenging due to the lack of suitable technology. Tn-glycopeptides have previously been discovered by applying VVA lectin or hydrazide chemistry for the enrichment of Tn-glycopeptides, followed by LC-MS/MS for site localization (Steentoft et al., 2011). Zheng et al. (2017) and You et al. (2018) make use of galactose oxidase to selectively oxidize Tn antigen glycans followed by enrichment using hydrazide beads and release with methoxylamine for MS analysis. Yang et al. (2020; and WO2021/041507A1) developed the EXoO-Tn method, which utilizes solid-phase immobilization of proteolytic peptides of proteins, modifying Tn by glycosyltransferase C1GalT1 and conversion of Tn to Gal(13C6)-Tn, released at the N-termino of the Ser/Thr residue by an endoprotease OpeRATOR, to yield site-containing glycopeptides. Although these existing methods further improved the knowhow in this complex technological area and report on a number of novel Tn antigens for therapeutic targeting, there is still a need for advancing glycoproteomic approaches in facilitating large-scale mapping of Tn-glycopeptide epitopes.

### BRIEF SUMMARY

This disclosure relates to means and methods for screening for novel O-linked glycans knowns as Tn- or Sia-Tn-antigens, particularly expressed on the cell surface of tumor cells. Several studies lead to the discovery of new O-glycan Tn-antigen epitope regions, against which antibodies can be developed for use as a therapeutic, or to apply as an in vitro diagnostic. In the disclosure, the first process steps are unique in that relate an in vitro method is applied wherein a specific combination of enzymatic treatments of glycoproteins and chemical labelling results in glycopeptides enriched for labelled (oxidized) GalNacs present at Ser or Thr of the protein backbone, and by using parallel tracks, providing samples for distinguishing Tn-antigen sites from SiaTn-antigen sites (FIG. 1). The innovative concept of this process stems from the particular oxidation of GalNacs derived from Tn antigen glycans, which is obtained by combining sialyltransferases used to block or protect free end-standing Galactoses prior to applying Galactose oxidase.

After digestion of the treated glycoprotein(s) into peptides, these labelled Tn-antigen-peptides may further be enriched by conjugation or immobilization on a solid phase specific for the conjugation of the label, such as tandem-mass-tag (TMT) labelling bound to anti-TMT resin. In particular, embodiments, the digestion step may be facilitated through the use of LysC, and trypsin, or alternatively, digestion with IMPa followed by LysC.

A second aspect of the disclosure further relates to a mass spectrometry process for analyzing and characterizing each of the Tn-antigen-peptides of interest. Also for this process, the specific combination of a discovery run using a triggered dual fragmentation approach, for characterization and quantification of the Tn-antigen-peptides, and optionally a second run for peptide identification and localization of the Tn-antigen site on the peptide (FIG. 4).

By combining both processes and starting from plasma-membrane enriched cell or tissue extracts (FIG. 7), including an internal standard for normalization, the large-scale identification of novel (Sia)Tn-antigen glycopeptide epitopes is in scope, with the ultimate goal of developing novel immunotherapy treatments.

### BRIEF DESCRIPTION OF THE DRAWINGS

The drawings described are only schematic and are non-limiting. In the drawings, the size of some of the elements may be exaggerated and not drawn on scale for illustrative purposes.

FIG. 1. Chemoenzymatic workflow for the labelling and enrichment of (Sia)Tn-antigens. Plasmamembrane-protein (PM) enriched proteomes of cells or tissue are loaded on a PVDF-coated, porous 96-well plate (a single glycoprotein is depicted). N-glycans are removed via PNGaseF digestion. Two paths can be followed, the left path includes a α2-3,6,8,9 neuraminidase step, which hydrolyzes all Sia and thereby transforms the SiaTn-antigens into Tn-antigens. This path will label and enrich both SiaTn- and Tn-antigens. The right path omits the neuraminidase treatment and will specifically label and enrich Tn-antigens. Free Galactoses are protected against Galactose oxidase (GaOX) based oxidation via sialylation by β-galactoside α2-3 sialyltransferase 1 (ST3Gal1) (T-antigen specific) and ST3 β-galactoside α-2,3-sialyltransferase 3 (ST3Gal3) (polyLacNAc specific). A subsequent one-pot reaction with GaOX and aminoxyTMT™ will label all unprotected end-standing Gal- and GalNAc-moieties. The excess of aminoxyTMT™ label is washed away and the glycoproteins are digested with LysC followed by trypsin or with IMPa followed by LysC and subsequently eluted from the plate. An extra TMT-enrichment step can be included with the immobilized anti-TMT™ antibody resin and subsequent elution by TMT™ elution buffer (both commercially available).

FIG. 2. Oxidation reaction of Galactose by Galactose Oxidase.

FIGS. 3A and 3B. Sialylation of the T-antigen by ST3Gal1/ST3Gal3 and the ability of Neu5Ac to protect Gal from being oxidized by GaOX. (FIG. 3A) A mix of GalGalNAc-Muc4 and GalNAc-Muc4 was incubated with ST3Gal1/ST3Gal3 and 200 μM CMP-Neu5Ac, which is a 100-fold excess compared to the glycopeptide concentration. Sialylation efficiency was measured by LC-MS at different timepoints. The left panel shows the relative AUC of the XICs of GalGalNAc-Muc4 and SiaGalGalNAc-Muc4, the right panel depicts the relative AUC of GalNAc-Muc4 and SiaGalNAc-Muc4. The specific activity of ST3Gal1 is >1000 μmol/min/μg, as determined by the supplier, 0.485 μg was added. ST3Gal3 has a specific activity of >400 μmol/min/μg, as determined by the supplier, 0.74 μg was added. N=3, mean±Standard error of the mean (SEM) is shown (FIG. 3B) The sialylated peptides (500 μmol SiaGalGalNAc-Muc4+500 μmol GalNAc-Muc4) were purified over C18-resin, pooled and incubated with 2U GaOX. The left panel shows the relative AUC of the XICs of sialylated GalGalNAc-Muc4 species, the right panel shows the GalNAc-Muc4 species.

FIG. 4. Flowchart of HCD-based mass-spectrometry method that allows identification and quantification of TMT-GalNAc modified peptides. The MS1 stage precursor scan applies a charge filter; followed by MS2 collision-induced dissociation (CID) fragmentation in the narrow m/z range [450-550] and at relative intensity above 20% to identify the 503.3 m/z and (optionally) 252.2 m/z peaks indicating the presence of labelled (Sia)Tn antigen, subsequently re-isolated for Higher energy Collisional Dissociation (HCD). The information on glycan localization will be lost due to the HCD fragmentation, but can be compensated by performing follow-up Parallel Reaction Monitoring (PRM) analysis on the same sample with, for instance, Electron-transfer dissociation (ETD), or Electron activated dissociation (EAD) fragmentation.

FIGS. 5A-5C. Optimization of the HCD-based MS method on a TMT-labelled, synthetic glycopeptide pool. A glycopeptide pool of 100 different Tn-glycopeptides was split in three equal parts and labelled with aminoxyTMTs, after which they were mixed in a 3:2:1 ratio (TMT126:127:128) and subsequently analyzed with the workflow depicted in FIG. 4. (FIG. 5A) The optimization of Normalized collision energy (NCE) for HCD fragmentation to obtain decent identification in combination with efficient reporter ion dissociation. A dual trigger method was used, where both charge variants of the TMT-GalNAc ion should be present for at least 5% in a narrow CID window. When these conditions were met, the labelled glycopeptide was fragmented via HCD with a NCE of 40%, 50% or 60% (all in separate runs). The number of unique IDs after performing a Mascot search (via Proteome Discoverer) is depicted on the y-axis, for three different FDR filter settings. For each of the NCEs, the matching quantification analysis is shown below the graph. Each datapoint represents a TMT-reporter ion ratio. The number of ratios per sample are depicted above the graphs. Red lines represent mean±SEM. (FIG. 5B) Comparison of two different trigger methods using the number of IDs from the TMT-GalNAc labelled glycopeptide pool. The settings of each trigger method are depicted below the graph, as well as the number of trigger events. For this experiment, the HCD spectra were generated with 40% NCE. (FIG. 5C) Comparison of HCD (40% NCE) with EThcD fragmentation on the number of IDs, ion scores and trigger events when the optimal trigger method is used: 503.3 m/z >20% intensity in a [450-550] CID window. FDR, False Discovery Rate.

FIG. 6. Comparison between the original EThcD-based method and the optimized HCD-based method. A glycopeptide pool of 100 different Tn-glycopeptides was split in three equal parts and labelled with different aminoxyTMTs, after which they were mixed in a 3:2:1 ratio (TMT126:127:128) and subsequently analyzed with two different MS/MS methods. The left part of the graph describes a method that is based on EThcD fragmentation for identification and glycan localization in parallel with HCD based fragmentation of the TMT-GalNAc ion (MS3) for relative quantification. The right part of the graph describes a method where relative quantification and identification are achieved by a single HCD fragmentation scan, without information for glycan localization. Each of the fragmentation cycles were triggered by the presence of TMT-GalNAc, detected in a fast CID ‘screening scan’. The number of unique IDs obtained with both methods is shown in the upper graph, with different FDR filter settings. The matching quantification analysis is shown below the graph. Each datapoint represents a TMT-reporter ion ratio. The number of ratio values are depicted above the graphs. Red lines represent mean±SEM. FDR, False Discovery Rate.

FIG. 7. Tn-Miner method workflow for discovery of novel O-glycopeptide cell surface epitopes. Samples composed of cell cultures or isolated tissue are lysed and Plasmamembrane (PM)-protein extracted using Invent Biotechnologies Kit, followed by immobilization on a solid phase, for example, a PVDF-membrane bottomed 96-well plate as described by Berger et al., 2015, to allow efficient buffer exchange and washing steps between each of the chemoenzymatic workflow for specifically (TMT) labelling of the (Sia)Tn-antigens of interest, which are finally analyzed for identification, localization and quantification using an innovative LC-MS/MS analysis on a high accuracy mass analyzer, such as an Orbitrap.

FIG. 8. O-glycan analysis of Erythropoietin produced in HEK293S WT vs HEK293S COSMC^{KO} cells. Intact protein LC-MS was performed on erythropoietin (EPO), produced in both WT (top panel) and COSMC^{KO} cells (bottom panel) after N-glycan removal with PNGaseF. The generated spectra were deconvoluted with BioPharma Finder and O-glycans were manually annotated.

FIG. 9. Optimization of Tandem Mass Tag (TMT)-labelling of Tn-EPO on solid phase. Tn-EPO was immobilized on different wells of a PVDF-coated 96 well plate and subsequently treated with PNGaseF. After N-glycan removal, the different wells were incubated with GaOX and aminoxyTMT (one-pot) in different reaction buffers (50 mM NaCitrate pH 6 or 50 mM NaPᵢ pH 7) at different temperatures (RT or 37° C.). Tn-EPO was digested on plate with Lysc and trypsin and eluted with 20% methanol, 0.1% TFA. After clean up with C18-resin, the samples were measured via LC-MS/MS. N=3, mean±SEM is shown.

FIGS. 10A and 10B. Surface exposed Tn-content of HEK293S WT vs COSMC^{KO} cells and the experimental set-up for the proof of concept experiment. (FIG. 10A) Screening of the surface exposed Tn-antigen content of both HEK293S WT and HEK293S COSMC^{KO} cells. The Tn-content was assessed via flow cytometry after staining of the intact cells with VVA-FITC, specific for the Tn-antigen. As control for aspecific binding, a control was taken along where VVA-FITC was preincubated with 400 mM GalNAc to block the glycan binding site. (FIG. 10B) Experimental set-up of the proof of concept experiment, PM-proteome enriched samples were divided over the wells of a PVDF-coated 96-well plate in duplicate. After completion of the chemoenzymatic workflow, the samples within each square were combined, and one of the combined samples was TMT-enriched via immobilized anti-TMT antibody resin. Each of the combined samples was analyzed via LC-MS/MS, with and without the trigger method developed in Examples 2 and 3, resulting in four different datasets from two different samples. The number depict the reporter ion mass of the TMT label for each well.

FIGS. 11A-11C. Performance of the chemoenzymatic workflow in combination with different MS/MS set-ups in terms of identification and quantification of the internal standard. The chemoenzymatic workflow was performed on HEK293SWT and COSMC^{KO} cells, in which Tn-EPO was spiked as internal standard. The workflow was performed as shown in FIG. 10, resulting in four different datasets from two samples. (FIG. 11A) Ion scores of all peptide-spectrum matches (PSMs) that match with the TMT-labelled O-glycopeptide of EPO. Two FDR filters are depicted. (FIG. 11B) XICs of all EPO peptides for both TMT-enriched and non-enriched samples. The indicated peptide is the TMT-labelled O-glycopeptide of EPO (SEQ ID NO:1). (FIG. 11C) Row normalized reporter ion intensities of the reporter ions that were associated with PSMs of the TMT-labelled O-glycopeptide of EPO with an FDR<5%. Normalization was performed by calculating the sum of all reporter ions for each PSM and dividing each reporter ion by this ‘row sum’. Mean±SEM is shown.

FIG. 12. Analysis of the triggered MS/MS workflow on the TMT-labelled O-glycopeptide of EPO (SEQ ID NO:1). Tn-EPO was spiked in both PM-enriched proteomes of HEK293S and HEK293S COSMC^{KO} cells before immobilization on a PVDF-plate. The experiment was performed as shown in FIG. 10. Here, the spectra from all different stages in the optimized triggered MS/MS workflow are shown for this internal standard peptide. The TMT-labelled O-glycopeptide of EPO was detected in MS1 (upper panel,*), and was re-isolated for MS/MS analysis with CID fragmentation in the Orbitrap. Here, the diagnostic TMT-GalNAc ion (**) was detected with a relative intensity over the threshold of 20%, within a narrow m/z range. This trigger resulted in a re-isolation of the precursor ion and fragmentation with HCD (40% NCE). This spectrum resulted in a PSM with the TMT-labelled O-glycopeptide of EPO and relative quantification via the reporter ions (zoom, squared region).

FIG. 13. Total ion current (TIC) chromatograms of the four datasets. The maximal TIC intensity was derived from a window of 80 to 120 min (dashed squares).

FIGS. 14A-14E. Evaluation of the Tn-Miner workflow on PM-enriched lysates of HEK293s WT and HEK293 COSMC^{KO} cells. (FIG. 14A) A porous, PVDF-coated 96 well plate was loaded as depicted here. 10 μg of the PM-enriched lysates were loaded per well, with addition of 100 fmol Tn-EPO as internal standard per well. The wells of each row were labelled with a different aminoxyTMT label, as indicated on the right side of the plate. (FIG. 14B) All PSMs that were identified as rank 1 with TMT-GalNAc as modification, are plotted in this graph. All reporter ion intensities were row normalized and linked per PSM. Channels 126, 127 and 128 are specific for HEK293s WT cells, channels 129, 130 and 131 are specific for the COSMC^{KO} channels. Missing values were imputed as 0, no FDR filtering is applied. The reporter ion series of all PSMs of TMT-Tn-EPO, the internal standard, are depicted in red. The Mean±SEM is depicted in yellow. N=328 (FIG. 14C) Numerical overview of the output generated by Proteome Discoverer with Mascot as search engine. All shown PSMs are rank 1, as determined by Mascot. Only ⅓^{th} of the total sample was injected for LC-MS/MS analysis, corresponding to 20 wells as input material (=200 μg of protein, including 1 μmol of Tn-EPO). (FIG. 14D) All HCD spectra that obtained at least one reporter ion are plotted in this graph. All reporter ion intensities were log 2 transformed and linked per spectrum. Channels 126, 127 and 128 are specific for HEK293s WT cells, channels 129, 130 and 131 are specific for the COSMC^{KO} channels. The reporter ion series of all PSMs of TMT-Tn-EPO, the internal standard, are depicted in yellow. (FIG. 14E) A typical spectrum from an HCD fragmented TMT-Tn-glycopeptide (SEQ ID NO: 2). The reporter ion series are shown in more detail on the right. *=TMT-Tn-ion (503.3 m/z).

FIGS. 15A and 15B. Repetition of the HEK293s WT Vs. COSMC^{KO} proof of concept experiment. (FIG. 15A) All HCD spectra that obtained at least one reporter ion are plotted in this graph. All reporter ion intensities were log 2 transformed and linked per spectrum. Channels 126, 127 and 128 are specific for HEK293s WT cells, channels 129, 130 and 131 are specific for the COSMC^{KO} channels. The reporter ion series of all PSMs of TMT-Tn-EPO, the internal standard, are depicted in yellow. (FIG. 15B) All spectra that contained all three COSMC^{KO} specific reporter ions and no WT specific reporter ions (yellow lines), as well as the spectra with at least a twofold increase in COSMC^{KO} specific reporter ion intensity compared to the WT reporter ions (black), were selected. From this elaborate list, 124 spectra were selected to generate a PRM list, based on their overall abundance and retention time. Reporter ion intensities of all 124 PRM candidates are depicted in this figure.

FIGS. 16A-16C. Evaluation of proteolytic activity of the glycoprotease IMPa on TMT labeled Tn-EPO. (FIG. 16A) Reported glycan specificity of IMPa. IMPa is a broad spectrum glycoprotease that hydrolyzes the amino acid backbone N-terminally from O-glycosylated Ser/Thr residues, if position-1 is different from Asp. Figure adapted from NEB. (FIG. 16B) Implementation of the IMPa enzyme in the Tn-miner workflow to obtain glycopeptides with a single, N-terminal glycan. (FIG. 16C) Evaluation of the hydrolysis efficiency of TMT-Tn-EPO by IMPa, in comparison to its natural substrate, being Tn-EPO (SEQ ID NOs: 3 and 4). After 3 h incubation with IMPa, LysC was added. XICs and AUC calculations were performed with Skyline.

### DETAILED DESCRIPTION

This disclosure will be described with respect to particular embodiments and with reference to certain drawings but the disclosure is not limited thereto but only by the claims. Any reference signs in the claims shall not be construed as limiting the scope. Of course, it is to be understood that not necessarily all aspects or advantages may be achieved in accordance with any particular embodiment of the disclosure. Thus, for example, those skilled in the art will recognize that the disclosure may be embodied or carried out in a manner that achieves or optimizes one advantage or group of advantages as taught herein without necessarily achieving other aspects or advantages as may be taught or suggested herein. The disclosure, both as to organization and method of operation, together with features and advantages thereof, may best be understood by reference to the following detailed description when read in conjunction with the accompanying drawings. The aspects and advantages of the disclosure will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the disclosure. Thus, appearances of the phrases ‘in one embodiment’ or ‘in an embodiment’ in various places throughout this specification are not necessarily all referring to the same embodiment but may.

### Definitions

Where an indefinite or definite article is used when referring to a singular noun e.g., “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the disclosure described herein are capable of operation in other sequences than described or illustrated herein. The following terms or definitions are provided solely to aid in the understanding of the disclosure. Unless specifically defined herein, all terms used herein have the same meaning as they would to one skilled in the art of the disclosure. Practitioners are particularly directed to Sambrook et al., Molecular Cloning: A Laboratory Manual, 4th ed., Cold Spring Harbor Press, Plainsview, New York (2012); and Ausubel et al., Current Protocols in Molecular Biology (Supplement 114), John Wiley & Sons, New York (2016), for definitions and terms of the art. Unless defined otherwise, all technical and scientific terms used herein have the same meaning as commonly understood by one of ordinary skill in the art (e.g., in molecular biology, biochemistry, structural biology, and/or computational biology).

The terms “protein” and “polypeptide” are interchangeably used further herein to refer to a polymer of amino acid residues and to variants and synthetic analogues of the same. The term “peptide” refers to a polymer of amino acid residues and variants and synthetic analogues of the same, wherein the peptide is of shorter length as compared to a protein or polypeptide, potentially as a result of protein digestion or proteolytic cleavage, preferably with a length of maximally 40-100 amino acids, most preferably not more than 50 amino acids. By “recombinant (poly) peptide” is meant a (poly) peptide made using recombinant techniques, i.e., through the expression of a recombinant or synthetic polynucleotide, which may be obtained in vitro and/or in a cellular context. When the chimeric (poly) peptide or biologically active (i.e., functional) portion thereof is recombinantly produced, it is also preferably purified, or isolated, as used interchangeably herein, or substantially free of culture medium, i.e., the impurities represent less than about 20%, more preferably less than about 10%, and most preferably less than about 5% of the volume of the protein preparation. By “isolated” or “purified” is meant material that is substantially or essentially free from components that normally accompany it in its native state. By “enriched” or “enrichment” is meant herein that material or specific components are present at a substantially higher amount as compared to the non-enriched material, so typically involving a purification or isolation step.

“Binding” means any interaction, be it direct or indirect. A direct interaction implies a contact between the binding partners. An indirect interaction means any interaction whereby the interaction partners interact in a complex of more than two molecules. The interaction can be completely indirect, with the help of one or more bridging molecules, or partly indirect, where there is still a direct contact between the partners, which is stabilized by the additional interaction of one or more molecules. The binding or association maybe non-covalent—wherein the juxtaposition is energetically favored by, for instance, hydrogen bonding or van der Waals or electrostatic interactions- or it may be covalent, for instance, by peptide or disulphide bonds.

As used herein, the term “sample” refers to any material that contains one or more (glyco)polypeptides, which can be a mixture of different polypeptides and further components. Sample is used in a broad sense herein and is intended to include a wide range of biological materials as well as compositions derived or extracted from such biological materials, as well as synthetic compositions. Biological samples may comprise, for instance, a cell suspension or cell culture, a body tissue or fluid, and may be derived from a subject, which may be a human or patient, providing thus for a clinical sample. The sample may or may not undergo preparation prior to applying the methods described herein. It may, for instance, be pretreated to achieve higher purity, a higher concentration of polypeptides, a lower concentration of contaminants. Non-limiting examples of such treatments include mechanical or chemical lysis of a cellular sample, or enrichment of a cellular (lysed) sample for the plasmamembrane fraction or components prior to application of the method.

“Surface”, “solid support”, “solid surface”, or “solid phase” as used interchangeably herein is a synonym for carrier or layer. The surface, support, carrier or layer may be a plate surface, a bead or a resin, and may be composed of several materials, including but not limited to nitrocellulose or other membrane materials, polystyrene, agarose, beaded polymers, or glass slides. The surface or support used for the method of the disclosure is suitable to use in enzymatic reactions, chemical treatment, detection of molecular labels, electrochemical signals, electromagnetic signals, and plasmon related events.

“Immobilization on a surface” or “affixing on a surface” or “immobilization on a support” or “conjugation on a support or solid phase” as used interchangeably herein refers to the attachment of one or more polypeptides to an inert, insoluble material, for example, a glass or PVDF surface resulting in loss of mobility of the polypeptides. For the methods disclosed in this disclosure, immobilization allows the polypeptide(s) to be held in place throughout the chemo-enzymatic treatment, or for specific enrichment of the labelled-Tn-antigen peptides. Proteins immobilized onto surfaces with high density allow the usage of small amount of sample solution. Many immobilization techniques have been developed in the past years, which are mainly based on the following three mechanisms: physical, covalent, and bioaffinity immobilization (Rusmini et al 2007 Biomacromolecules 8:1775-1789; U.S. Pat. No. 6,475,809; WO2001040310; U.S. Pat. No. 7,358,096; US20100015635; WO1996030409; WO2013112745).

Mass spectrometry is an analytical tool that can be used to determine the molecular weights of chemical compounds such as (poly) peptides by generating ions and separating these ions according to their mass-to-charge ratio (m/z). The ions are generated by inducing either a loss or a gain of a charge by the chemical compounds, such as via electron ejection, protonation, or deprotonation. The ions are then separated according to their m/z values and detected. The resulting data are often presented as a spectrum, a two-dimensional (2-D) plot with m/z ratio on the x-axis and abundance of ions on the y-axis. Thus, this spectrum shows the distribution of m/z values in the population of ions being analyzed, allowing to identify a given compound in a sample. Practitioners are more specifically directed to Gross, Mass Spectrometry: A Textbook, 3rd ed., Springer-Verlag GmbH (2017), for definitions and terms used in mass spectrometry.

Glycosylation sites and glycan structures present on proteins, referred to herein as glycoproteins, can be determined in part by enrichment of glycopeptides, or by enzymatically or chemically releasing glycans. The structure of released glycans and remaining peptides can be determined by mass spectrometry and liquid chromatography/mass spectrometry. Fragmentation techniques can be used to obtain glycan structures and amino acid sequences of the peptide backbone of glycopeptides with mass spectrometry, though often a low fragmentation efficiency is obtained with glycopeptides, and customized approaches are often desired to a certain analytical setting. Practitioners are particularly directed to Lauc and Wuhrer, High-Throughput Glycomics and Glycoproteomics, Springer Science+Business Media, LLC, part of Springer Nature, Humana New York, NY (2017), for definitions and terms used in glycobiology and glycoproteomic approaches.

### DETAILED DESCRIPTION

This disclosure relates to a novel methodology for discovery of novel unknown glycosylation sites of O-linked Tn-antigen or STn-antigen glycans on the surface of cancer cells, to ultimately provide unique, highly selective epitopes for targeting specific tumor types.

The “Tn antigen” is a carbohydrate antigen defined as one N-acetylgalactosamine (GalNAc) monosaccharide in an O-glycosidic alpha-linkage individually linked to the amino acids threonine, serine or more rarely tyrosine (GalNAc—O-Ser/Thr). Tn antigen is normally not expressed in peripheral tissues or blood cells. The Tn antigen is abnormally expressed in many human carcinomas and expression correlates with metastasis and poor survival. Expression of this antigen in a majority of human carcinomas of all types arises from a blockage in the normal O-glycosylation pathway in which glycans are extended from the common precursor GalNAc—O-Ser/Thr (In antigen). Under normal conditions, the precursor GalNAc—O-Ser/Thr is extended and further modified to normal O-glycans by T-synthase (C1GALT1) to form core 1 O-glycans. Biosynthesis of active T-synthase requires its molecular chaperone, Cosmo. Conditions that cause genetic and epigenetic silencing of T-synthase and/or Cosme (e.g., cancer) can lead to expression of the precursor Tn antigen.

The sialyl-Tn antigen (Neu5Acα2-6GalNAcα-O-Ser/Thr), known as STn or SiaTn, is a truncated O-glycan containing a sialic acid α-2,6 linked to GalNAc a-O-Serine/Threonine (Ser/Thr). Both Tn and STn antigens have been reported to be biomarkers of various cancers and are associated with an adverse outcome and poor prognosis in cancer patients. A major obstacle in detecting Tn antigen in circulating glycoproteins is that Tn antigen can also be found on a subset of glycoforms of human IgA1, within the hinge region. Another complication is that anti-Tn reagents that bind terminal linked GalNAc residues, as found in blood group A (BGA) and the Forssman-related antigens, may interfere with specific detection of Tn antigen on tumor-derived glycoproteins. Thus, a useful anti-Tn antibody for cancer diagnostics, research, and therapeutics would specifically recognize abnormal Tn antigen, and not cross-react with Tn on IgA1, or normal glycans terminating in GalNAc. There is still a need for those (S)Tn-antigen-specific binders that specifically recognize those O-glycans present on glycoprotein sites specifically present on tumor cells.

Our present method allows to identify those (S)Tn-antigen-peptide epitopes specific for carcinoma cells by providing for large-scale throughput possibilities in combination with the high selectivity obtained by implementing a chemo-enzymatic workflow followed by an innovative customized LC-MS/MS analysis process. So, in a first aspect, the method described herein is highly selective by first subjecting the glycoprotein sample to a treatment wherein the GalNacs of a Tn-antigen are specifically labelled without labelling non-Tn-antigen undesired Gal or GalNac glycan residues present elsewhere on the glycoprotein. This is achieved by integration of a ‘protection’ step during which enzymatic treatment using sialyltransferases specific for those ‘undesired glycans’ is used in the pretreated, more specifically, using a sialyltransferase specific for Galactose of Gal-β1,3-GalNAc in core 1 or core 2 type glycans, and sialyltransferase specific for end-standing Galactose within polyLacNAc, and a sialyltransferase-substrate, for protecting the free galactoses against oxidation. In the exemplified method, this protection step is performed using a combination of ST3Gal1 and one or more of ST3Gal3, ST3Gal4, and/or ST3Gal5. The pretreatment is then followed by incubation with Galactose oxidase and oxime-based labelling via the oxidized residues.

In a specific embodiment, the method initially provides the glycoprotein sample in solution. In an alternative embodiment, the glycoprotein sample is immobilized on a surface or solid phase or solid support, such as a plate, more specifically, a PVDF coated plate, or a resin or a bead.

In a further embodiment, it is desired to remove the N-glycan structures present on the glycoproteins in the sample prior to the protection step, as to simplify later analysis, such as mass spec analyses. The removal of N-glycans on the Asparagine residues of the glycoproteins in the sample may be removed using a PNGase F treatment, or alternatively, other endoglycosidases such as Endo F and EndoH may be applied. Endo F1 cleaves high mannose and some hybrid type N-glycans; Endo F2 removes biantennary and high mannose; Endo F3 releases of triantennary and fucosylated biantennary N-glycans; and Endo H removes hybrid or high mannose glycans.

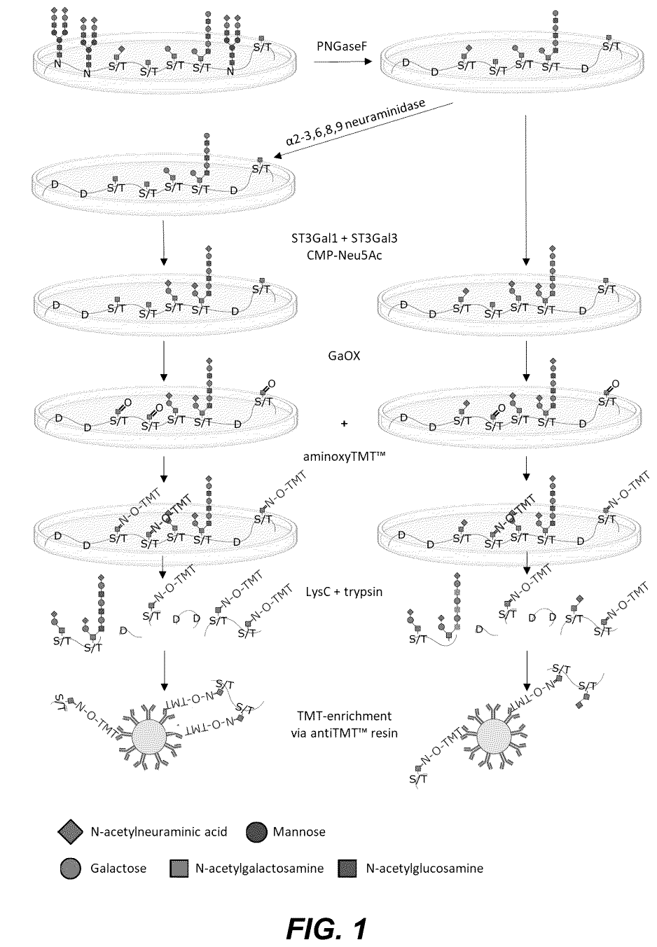

In a further specific embodiment, the chemo-enzymatic method as described herein includes an additional step, so after removal of the N-glycans on the glycoproteins in the sample, the sample is first treated to hydrolyze or desialylate the sialylated-residues by applying a sialidase, a neuraminidase or if preferred to do a chemical treatment, hydrolysis can be done, for instance, by using TFA. As a result, the further workflow, with next the protection step and then the GaOx and simultaneous labelling step, will provide for the labelled GalNac-peptides indicative of the glycosylation sites with Tn- and STn-antigen presence (see also FIG. 1 the left path). The sialidase or neuraminidase enzymatic reaction can be performed in wide range of buffers and temperatures. In a specific embodiment, treatment with, for instance, 0.1% TFA treatment at 75° C. for 1 hour is performed to remove sialic acid.

Following the enzymatic treatments of the (immobilized) glycoprotein sample, the glycoproteins are envisaged to be digested into peptides for further analysis, which is a step that can be performed using, for example, different enzymes including, but not limited to, trypsin, endoproteinase Lys-C, Lys-N, Arg-C, Asp-N, clostripain, exopeptidase, carboxypeptidase, cathepsin C, cyanogen bromide, GluC, chymotrypsin, papain, Pepsin, Proteinase K, subtilisin, and thermolysin, IMPa or combinations of multiple enzymes can be used to digest the proteins into peptides. The digestion reaction can be performed at room temperature or 37° C. or any temperature above 0° C.

Finally, after obtaining the labelled digested peptides, an enrichments step of the labelled peptides is required on label-specific binding agent, wherein the agent can be provided on a solid support, a bead, a plate, or alike, simplifying the enrichment of the labelled components in the reaction mixture. In a specific embodiment, the peptides are labelled with aminoxy Tandem-mass-tag (TMT) label resulting in aldehyde groups, since TMT isobaric tags are specifically useful for quantification of the glyco-peptides in LC-MS/MS, and the enrichment step is obtained using commercially available anti-TMT resin. In a particular embodiment, removal of the enriched labelled glycopeptides is further also obtained using a commercially available release reagent. In cases where labelling or conjugation of oxidized carbohydrate/glycoprotein samples is desired without the need for TMT and follow-on MS analysis, alternatives are known to the skilled person.

For enrichment, the Tn-antigen-peptides can be conjugated to any solid-phase. In certain embodiments, the enriched Tn-glycopeptides are conjugated to beads through amine and aldehyde reduction, and in a specific embodiment, anti-TMT resin is applied.

A second aspect of the disclosure relates to a liquid chromatography-tandem mass spectrometry (LC-MS/MS) method to identify, quantify, and localize the(S) Tn-antigen-peptide epitopes on the glycoprotein of the sample, comprising the steps of:
apply a labelled glycopeptide-containing sample to an MS1 precursor scan on a high accuracy mass analyzer, wherein the label preferably is an isobaric label, more preferably a TMT-label,
follow the precursor scan with MS2 collision induced dissociation (CID) fragmentation, using a narrow m/z range with a trigger for m/z ions specific for the (labelled) Tn-antigen-peptide, triggering re-isolation of the precursor ion as of at least 20% relative ion intensity,
perform higher energy collision dissociation (HCD) fragmentation to the reisolated precursor ions at elevated normalized collision energy, for releasing of reporter ions and relative quantification and identification of the glycopeptides.

The method thus allows to map the(S) Tn-antigen peptides, which are obtained, for instance, through the chemo-enzymatic workflow described herein. Preferably, the Tn-antigen peptides are thus obtained from a glycoprotein sample, by way of enzymatically treating the glycoprotein sample to remove the N-glycans present on the glycoproteins, preferably using peptide: N-glycosidase F (PNGAseF), followed by sialylating the O-glycans on the glycoprotein using a sialyltransferase specific for Galactose of Gal-β1,3-GalNAc in core 1 or core 2 type glycans, and a sialyltransferase specific for end-standing Galactose within polyLacNAc, and a sialyltransferase-substrate, and further oxidizing the O-linked GalNac and Gal residues using Galactose oxidase (GaOx) and simultaneously label these oxidized glycans, and optionally, though preferably in preparation of the LC-MS/MS mapping: digesting the glycoprotein into peptides, and/or enriching the labelled Tn-peptides by conjugation on a label-specific binding support.

So, in a specific embodiment, the labelling is performed using an isobaric label, more preferably TMT-label.

With ‘mapping’ of the(S) Tn-antigen peptides is meant that one or more of the following properties are clarified for the particular(S) Tn-antigen(s) under analysis: i) the position of the Tn-antigen on the peptide (or localization) is revealed, (ii) the quantification of the number of Tn-antigens present on a peptide or protein/in a sample, (iii) the identification of the surface epitope or surface protein is determined.

In a further specific embodiment, the method in step a. applies a charge filter of ≥3+ or ≥2+. A further specific embodiment relates to the method wherein step b. comprises a narrow m/z range of 200-2000, or of 250-1500, or of 350-2200, or of 350-1500, or of 200-1000, or of 200-800, or of 200-600, or of 200-550, or of 350-1000, or of 350-800, or of 350-600, or of 350-550, or of 450-550. In a further specific embodiment the m/z ions used as a trigger representing the glycopeptide with Tn-antigen label of interest are provided by the ion of 503.3±0.5 m/z, and/or 252.2±0.5 m/z.

In a specific embodiment, with elevated NCE in step c. is meant at least 20%, or at least 30%, or at least 40%, or at least 50%, or at least 60%, with in a preferred embodiment 40% or more.

The LC-MS/MS method described herein provides detection by a discovery run using a three-stage mass spectrum, wherein the first stage comprises a precursor scan (MS1) of the glycopeptide sample. In a second stage, data acquisition is a mass analysis scan (MS2) obtained after collision induced dissociation (CID) of the glycopeptides. In a third stage, data acquisition is a mass analysis scan (MS2) obtained after higher energy collision dissociation (HCD), or a derivative fragmentation method thereof, such as known in the art, and, for instance, but not limited to stepped collision energy HCD (sceHCD), of the reisolated precursor ions.

In parallel to the latter stage, the method may further comprise a step d. with a second LC-MS/MS run wherein a trigger list is built up from the reisolated precursor ions in step c of the first run, for a PRM analysis using ETD, Electron-transfer dissociation with supplemental higher energy collisional dissociation (EThcD), or EAD fragmentation.

This arrangement toward the second run advantageously provides the option for localization of the (S)Tn-antigen glycosylation site on the peptide, especially when multiple sites are present on the peptide.

In a further specific embodiment, the method as described herein is applied on a high accuracy mass analyzer such as an Orbitrap Fusion™ Lumos™ Tribrid™ mass spectrometer, and/or the second run in step d. is performed on an Orbitrap Fusion™ Lumos™ Tribrid™ when ETD or EThcD fragmentation, or a ZenoTOF 7600 when EAD fragmentation, is used, resp.

The following examples are intended to promote a further understanding of the disclosure. While the disclosure is described herein with reference to illustrated embodiments, it should be understood that the disclosure is not limited hereto. Those having ordinary skill in the art and access to the teachings herein will recognize additional modifications and embodiments within the scope thereof. Therefore, the disclosure is limited only by the claims attached herein.

### EXAMPLES

### Introduction

This disclosure relates to a novel innovative method for discovery of O-glycopeptide cell surface epitopes by selectively label and enrich for (Sia)Tn-antigen-peptides, which are abundantly present on carcinoma cells. Through large-scale application of the present method, novel highly specific glycol-peptide epitopes are provided for improving specificity of immunotherapy treatments.

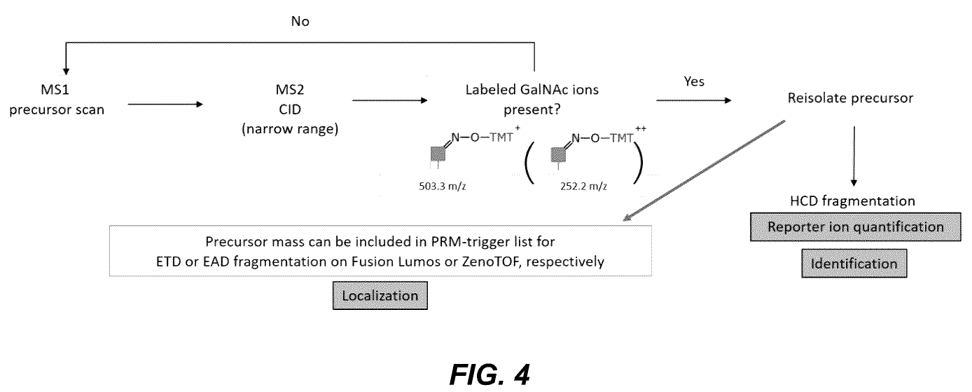

The Tn-miner methodology as presented herein (FIG. 7) is applicable to cell culture as well as tissue samples, and makes use of a combined workflow based on chemo-enzymatic treatments and an innovative LC-MS/MS analysis as reported herein for the first time. Samples containing cell surface glycoproteins are first subjected to lysis and plasmamembrane (PM)-enriched extracts are prepared, which may subsequently be immobilized on a solid phase to allow efficient buffer exchange and washing steps between each of the newly developed and optimized chemoenzymatic workflow as outlined in FIG. 1, and discussed in Examples 1 and 5. The resulting mixture of enriched TMT-labelled Tn-antigen glycopeptides are subjected to LC-MS/MS analysis (FIG. 4), which was developed (Example 2), and further improved to allow maximal throughput for selective antigen identification, quantification and localization on a high accuracy mass analyzer (Example 3). Furthermore, the addition of a profoundly characterized internal standard (Tn-glycoprotein) immediately prior to conjugation of the glycoprotein sample to the solid phase (as shown in Example 4) allows to track the TMT-conjugation efficiency, and to normalize the results for relative quantification, which is needed to correct for possible variation in the yield of TMT-labelled Tn-glycopeptides per sample or well of a PVDF-coated 96-well plate (further referred to as the “well-effect”). This “well-effect” is caused by potential variation in each well in terms of binding capacity, chemoenzymatic efficiencies, material loss during washing steps and possible variability in elution efficiency. Finally, the proof of concept using PM-enriched lysates of HEK293S WT and HEK293S COSMC^{KO} cells, spiked with the EPO internal standard, were analyzed with the chemoenzymatic workflow in a sixplex set-up, with and without TMT-enrichment step, and analyzed using the optimized LC-MS/MS setup (Example 6-7).

### Example 1. Galactose Oxidase (GaOX)-Based Chemoenzymatic Workflow

A representation of the complete chemoenzymatic workflow depicting a single, immobilized glycoprotein is shown in FIG. 1. This workflow describes a path for Tn-detection only (right path, FIG. 1), and one for combined detection of SiaTn-ant Tn-modified peptides (left path, FIG. 1). The latter includes a neuraminidase treatment after the PNGaseF digest, which transforms all SiaTn-antigens into Tn-antigens. Both paths can be performed in parallel, after which the identified epitopes and their quantitation can be compared between the two samples to deconvolute the relative occupancy with Tn or SiaTn. For instance, epitopes that are identified in the neuraminidase treated sample but are absent in the untreated sample will likely be SiaTn-antigens.

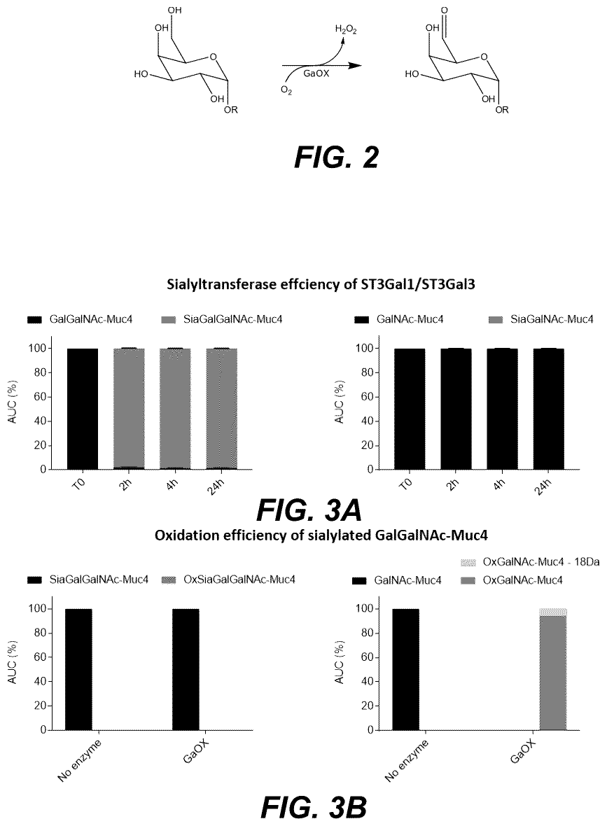

After removal of all N-glycans via PNGaseF and (optional) removal of Neu5 Ac with α2-3,6,8,9 neuraminidase, all end-standing, non-target Gal-moieties are sialylated in order to protect them against oxidation by galactose oxidase (GaOX) (FIG. 2). Indeed, β-galactoside α2-3 sialyltransferase 1 (ST3Gal1) transfers the Neu5Ac-moiety of CMP-Neu5Ac to the Gal of Gal-β1,3-GalNAc in core 1 or core 2 type glycans (Kitagawa & Paulson, 1994; Ortiz-Soto et al., 2019; Gupta et al., 2016). In order to protect all end-standing Gals in all mucin-type O-glycan structures against GaOX, ST3Gal1 should here be accompanied with other sialyltransferases that have specificity for end-standing Gals within polyLacNAc or Lewis structures. ST3Gal3, ST3Gal4 and ST3Gal5 are described to sialylate the end-standing Gal within these glycans, while core 1 and 2 glycans are no substrates for these STs (Gupta et al., 2016). ST3Gal3 was tested in a first round of experiments, although no synthetic substrate containing polyLacNAc structures is currently available in the lab to specifically test ST3Gal3 efficiency.

Sialylation efficiency of Galβ1,3-GalNAcα-Muc4 (a synthetic glycopeptide, produced in-house) by the ST3Gal1/ST3Gal3 cocktail was evaluated in a time kinetic experiment (FIG. 3A), at a 120-fold enzymatic activity excess of ST3Gal1 when incubated for 2 h and a CMP-Neu5Ac concentration of 200 μM (i.e., 100-fold molar excess over substrate). The KM-value of ST3Gal1 for CMP-Neu5Ac is described to be 8.5 μM (Jeanneau et al., 2004). As internal control, GalNAcα-Muc4 was spiked into the same sample, to assess the possible, unwanted sialylation of the Tn-antigen. After two hours of incubation, 98% of sialylation was observed. Longer incubation periods did not further increase sialylation of Galβ1,3-GalNAcα-Muc4. No sialylation of GalNAcα-Muc4 was observed (FIG. 3A). This concentration of CMP-Neu5Ac was also tested with ST3Gal1 alone, and the same sialylation efficiency was observed as depicted in FIG. 3A (data not shown).

The sialylated peptides (500 μmol) were eventually purified over C18 resin and incubated with 2U GaOX. In this approach, GaOX from Fusarium graminearum is deployed to oxidize all end-standing Gal and GalNAc moieties of O-glycans in order to create a reactive aldehyde function at their C6 position. GaOX is a radical copper oxidase that oxidizes the C6 position of Gal and GalNAc by transferring two hydrogen atoms to oxygen as electron acceptor, forming hydrogen peroxide (Avigad et al., 1962; Whittaker 2005) (FIG. 2). Happily, no oxidation was observed for the sialylated Galβ1,3-GalNAcα-Muc4 samples, while the internal control of GalNAc-Muc4 was completely oxidized (FIG. 3B). In conclusion, α2,3-sialylation completely protects Gal from being oxidized by GaOX.

### Example 2. Development of a Mass Spectrometry Method Using a Synthetic Glycopeptide Pool

The end product of the Tn-Miner chemo-enzymatic workflow as shown above is a mixture of TMT-labelled glycopeptides, where the TMT label is covalently linked to the C6 of GalNAc via an oxime bond. TMT labels allow for relative quantification in a multiplex set-up, sixplex in the case of aminoxyTMT™. Relative quantification is achieved through the release of the reporter ions from the isobaric TMT labels. These reporter ions differ in mass due to a different distribution of heavy, stable isotopes of C and N within the whole TMT molecule. To release these reporter ions, quadrupole fragmentation is required in combination with a high accuracy mass analyzer e.g., an Orbitrap. Ion trap analyzers cannot be used for this type of quantification due to their lower accuracy (compared to Orbitraps) and due to the so-called one-third rule, which states that the lowest detectable m/z value in an ion trap will be ca. 30% of that of the precursor m/z (Want et al., 2005; Griffin et al., 2007). The low m/z values of the reporter ions (126-131 Da) will therefore be undetectable for many fragmented peptide ions when using an ion trap.

Precursor scanning is thus performed in the Orbitrap, followed by CID fragmentation of the most intense peaks in the ion trap. With CID fragmentation, the TMT-GalNAc ion (503.3 m/z) is expected to be one of the most intense ions, due to the positive charge that is present on the TMT-label and the fact that the labile O-glycosidic bond has the highest probability to get fragmented. Ion trap-based CID was chosen to take advantage of the fact that fragmentation in CID is specifically tuned for the particular m/z value of the isolated ions. The frequency of the alternating current is chosen to specifically resonate the trapped ions, based on the precursor m/z. As a consequence, produced fragments are not fragmented any further and the easily lost 503.3 m/z fragment will be present with high intensity. Whenever a mass of 503.3 m/z is detected upon CID, the Orbitrap Fusion™ Lumos™ Tribrid™ mass spectrometer is tuned to automatically re-isolate the same precursor ion as to follow EThcD fragmentation, with readout in the ion trap for identification. In parallel, the 503.3 m/z ion is reisolated for HCD based fragmentation (MS3) and readout in the orbitrap for quantification. This 503.3 m/z trigger-based method was expected to limit the blind spots of the mass spectrometer. Moreover, the CID read out is fast, enabling the fragmentation of a high number of precursors, resulting in a sensitive analysis.

This workflow was tested on a pool of 100 synthetic glycopeptides, all bearing a Tn-antigen on a defined Ser or Thr. This glycopeptide pool was labelled with the TMT-conjugation protocol. The intensity threshold of the trigger m/z was empirically assessed by analyzing CID fragmentation spectra of TMT-GalNAc-Muc4, generated on a LTQ Orbitrap XL mass spectrometer, where the most intense fragment ion was always represented by the 503.3 m/z ion of TMT-GalNAc (data not shown). For these pilot experiments on the glycopeptide pool on the Orbitrap Fusion™ Lumos™ Tribrid™, an intensity threshold of 80% was chosen. The raw data files that are generated are complex and contain CID, HCD and EThcD spectra. Both CID and EThcD spectra were separated by ProteomeDiscoverer (PD) and subsequently searched with Mascot against the human protein database (Swissprot, version of January 2021) with the addition of a concatenated sequence made from the sequences of the glycopeptide pool as a new, synthetic protein. The MS3 HCD spectra were analyzed via the ‘Reporter Ions Quantifier’ node in PD and linked to the PSMs derived from the EThcD that were derived from the same precursor ion.

From the test experiments on the glycopeptide pool, it was concluded that a charge filter of ≥3+ on MS1 level was more selective for TMT-labeled glycopeptides, due to the extra charge originating from the TMT-label. An increase in charge state allowed for a reduced m/z range for the precursor scan, with a max. m/z value of 1500. This lower range decreases the MS1 cycle time, again limiting blind spots.

The CID spectra that were linked to these precursor ions were checked for the presence and intensity of the 503.3 m/z peak. This peak was present in all CID spectra, although not always reaching 80% of the maximum intensity value. Also, a 252.2±0.5 m/z ion was often detected in addition to the 503.3 ion, which corresponds to the double charged TMT-GalNAc ion. Two different options are thus possible for an improved trigger method: (i) Both ions (503.3 and 252.2 m/z) should be present in the CID spectrum, without stringent intensity filter, since there will only be a minor chance that this ion combination occurs by chance, or (ii) lowering the intensity threshold of the 503.3 m/z peak, since the double charged variant was not always detected. Trigger option (i) should result in a very specific trigger method, which might miss certain labelled peptides that lack a 252.2 m/z ion, while option (ii) might be less selective although more sensitive since more TMT-GalNAc containing peptides might be included. To boost the relative intensity of the trigger ions, the m/z range for CID fragmentation was narrowed to [200-550] m/z if both triggers are used, or to [450-550] m/z if only 503.3 is used as trigger. Next to a boost in sensitivity for the reporter fragments, this should also result in faster CID acquisitions, reducing cycle times.

Besides a too stringent trigger method, a high variability was noticed in the quality of the EThcD spectra of the different glycopeptides. Some of the unidentified peptides in the earlier described method triggered the mass spectrometer but resulted in low quality EThcD spectra that were highly dominated by the precursor ion in combination with their charge-reduced derivatives. In these spectra, close to no fragment peaks were observed, resulting in a lack of identification. To solve this issue, HCD fragmentation was tested at elevated normalized collision energy (NCE, ≥40%) (instead of EThcD fragmentation). Because of the TMT label, an elevated CE should be sufficient for efficient fragmentation, since efficient reporter-ion release demands for higher collisional energies. Switching to full HCD fragmentation also avoids problems with data analysis due to the mixture of TMT-GalNAc removal (=neutral loss) that affects b- and y-ions and non-neutral loss affected c- and z-ions. This phenomenon decreases the scoring of the PSM in most frequently used search engines (Mascot in this case) since a neutral loss cannot be specified, separately for every fragment ion type. HCD fragmentation only will result in loss of information on the localization of the TMT-GalNAc-moiety, unless only one Ser or Thr is present in the amino acid sequence. An elevated NCE should result in efficient release of the reporter ions, allowing for relative quantification within the HCD spectrum. If a peptide triggered the mass spectrometer for HCD fragmentation and the expected reporter ions are present in its spectrum, it should be possible to conclude that this peptide was modified with a TMT-GalNAc moiety. The precursor m/z values of these identified peptides (or only the ones of which quantification identifies them as promising biomarkers for the tumor under study) can then be listed in a trigger list for parallel reaction monitoring (PRM) analysis during a second LC-MS/MS run of the sample on an Orbitrap Fusion™ Lumos™ Tribrid™ mass spectrometer with ETD fragmentation or on a ZenoTOF 7600 mass spectrometer with EAD fragmentation in order to localize the TMT-GalNAc moiety (FIG. 4).

### Example 3. Optimization of a HCD-Based MS Method Using a Synthetic Glycopeptide Pool

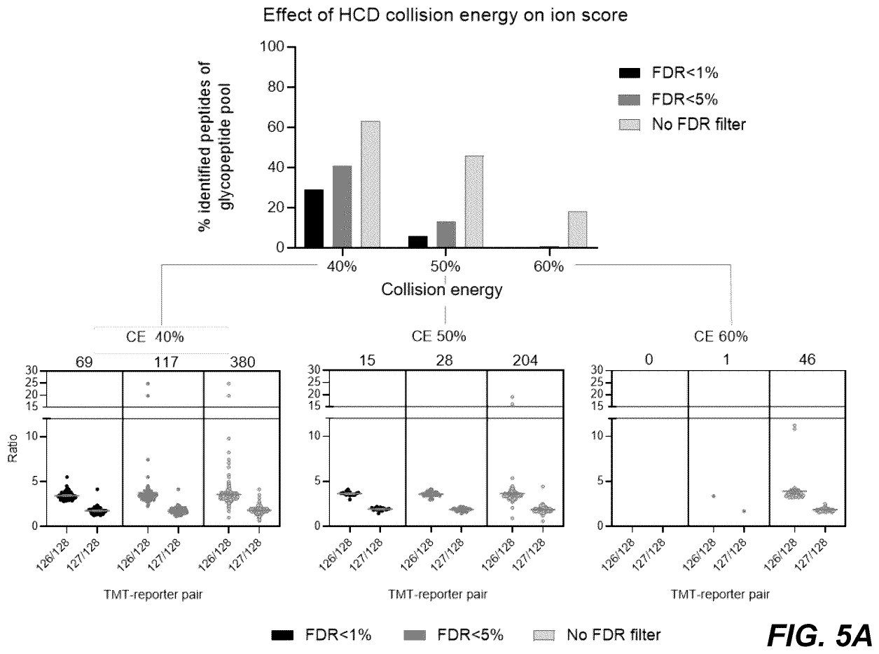

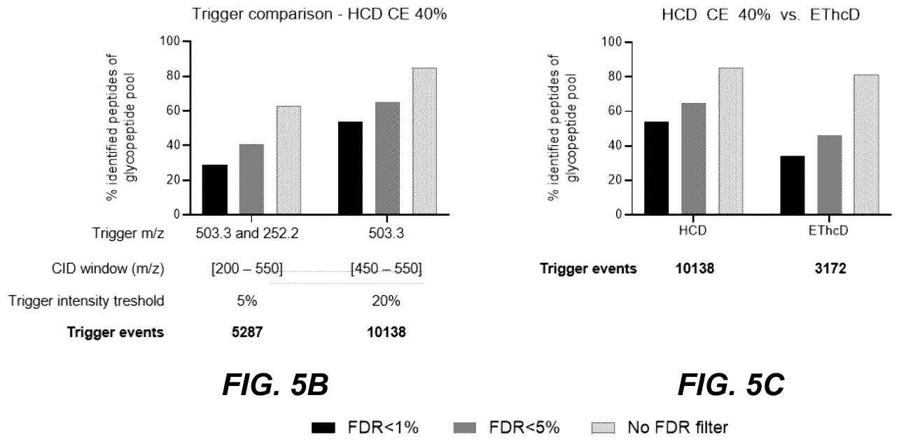

In a first optimization experiment, the optimal HCD NCE was assessed for proper identification and quantification. For this experiment, a peptide pool was labelled with three different TMTs (TMT126, 127 and 128) and mixed in a 3:2:1 ratio. Peptides were analyzed by MS using the method shown in FIG. 4. As a trigger, the dual trigger method was chosen, where both TMT-GalNAc ions should be present each with a minimal relative intensity of 5%. This percentage was chosen to induce trigger events with high sensitivity, while avoiding trigger events from the noise. The same sample was analyzed three times, each time with a different NCE for HCD: 40, 50 and 60% (FIG. 5A). A 40% NCE resulted in the most IDs and 29 glycopeptides with FDR<1%, 41 with FDR<5% and 63 glycopeptides without FDR filter were able to be identified. The mean reporter ratios of the NCE 40% sample matched the expected values, again with very narrow SEM (FIG. 5A). Thus, HCD fragmentation with 40% NCE results in proper quantification and identification.

The effect of the trigger method on the identification performance was tested by reanalyzing the same sample with the workflow depicted in FIG. 4, with 503.3 m/z as single trigger ion with a relative intensity of 20% within the narrow CID window (FIG. 5B). An increase in background intensity was expected due to this very narrow mass window and therefore the trigger threshold was increased from 5% to 20% relative intensity. The mass spectrometer was triggered 10138 times with this method, compared to 5287 triggers with the dual trigger method. This increase was translated to an increase in IDs. A total of 54 and 65 IDs were obtained with an FDR filter of <1% and <5%, respectively, and 85 IDs when no FDR filter was applied (FIG. 5B). To have a direct comparison on EThcD fragmentation versus HCD fragmentation, the same sample was analyzed with the optimal trigger settings (503.3 m/z, >20% relative intensity) and fragmented with EThcD. This comparison shows that if no FDR filter is applied, the number of IDs for EThcD and HCD are very close to each other: 81 and 85, respectively. However, a profound difference is observed when FDR filters are applied as about 20 more IDs are achieved for the HCD-generated spectra as compared to the EThcD-generated spectra with both FDR filters. In other words, there is a clear increase in the ion scores obtained via HCD compared to EThcD. The number of trigger events is over three times lower for the EThcD-based workflow compared to the one based on HCD, likely due to the longer cycle times needed for EThcD and occupation of the ion trap, limiting the number of CID spectra that can be generated (FIG. 5C).

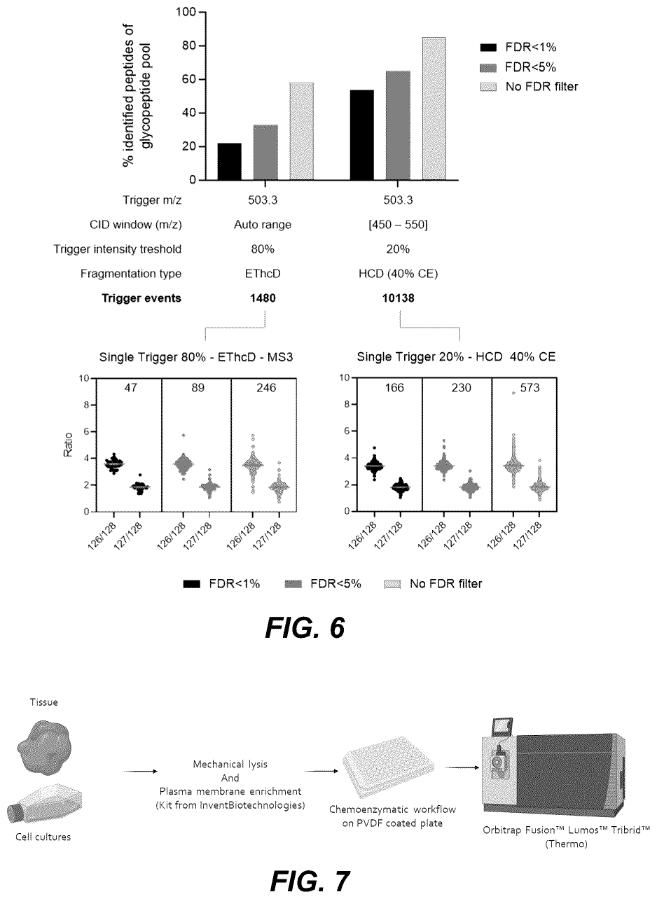

In a final experiment, the new optimized trigger and fragmentation method (503.3 m/z trigger in narrow CID window with an intensity above 20% in combination with HCD) was compared with the original method (503.3 m/z trigger in a broad CID window with intensity above 80% in combination with EThcD) (FIG. 6). A clear increase in IDs was observed for all different FDR filters with the new method. The mean reporter ion ratios and their SEM-values do not differ between the old and the new MS method, although there is a clear increase in the number of datapoints using the new method due to the increase of the number of trigger events (FIG. 6).

So the optimized triggered HCD-based MS workflow was demonstrated to allow identification of 65 TMT-Tn-labelled glycopeptides with an FDR<5%, out of a synthetic peptide pool of 100 Tn-glycopeptides, after GaOX/aminoxyTMT™-conjugation. This optimal set-up included a CID-based scan for the 503.3 m/z reporter ion, within a narrow mass range of [450-550] m/z, and relative reporter ion intensity of at least 20%. When triggered, the precursor ions were reisolated for HCD-fragmentation with successful identification and glycan localization and quantification of TMT-GalNAc-containing glycopeptides in a single run (FIG. 4).

The increase in ion scores that is observed for HCD-generated spectra is mainly a result of the better fragmentation efficiency of HCD (compared to EThcD), in combination with the follow-up search applying a commonly used search engine, like Mascot (alternatives are provided by Andromeda (MaxQuant), MSFragger and Sequest). The TMT-GalNAc modification will be removed upon CID/HCD and the search engine treats it as a neutral loss type of modification to all types of fragment ions. So, for the analysis with Mascot, the neutral loss setting was applied for all HCD-generated spectra.

An optimal ion scoring was prioritized using with frequently used search engines by applying HCD (instead of EThcD), although this results in the loss of glycan localization in case a peptide holds more than one possible glycosylation site. The latter can be compensated by reanalyzing the same sample in PRM mode on an Orbitrap Fusion™ Lumos™ Tribrid™ mass spectrometer with ETD or EThcD fragmentation or on a Zeno TOF 7600 mass spectrometer with EAD fragmentation, this to localize the TMT-GalNAc moiety (Baba et al., 2022). The trigger list that should be used for such PRM experiments thus contains all m/z values of biomarkers of interest that triggered an HCD event in the discovery run and of which the HCD spectra contained the expected reporter ions. This combination of events makes it very likely that the precursor ion was in fact from a Tn-bearing glycopeptide. In combination with charge and the retention time, this should be sufficiently discriminative.

### Example 4. O-glycosylation Profiling of Tn-glycoprotein Erythropoietin Produced in HEK293S COSMC^{KO} Cells

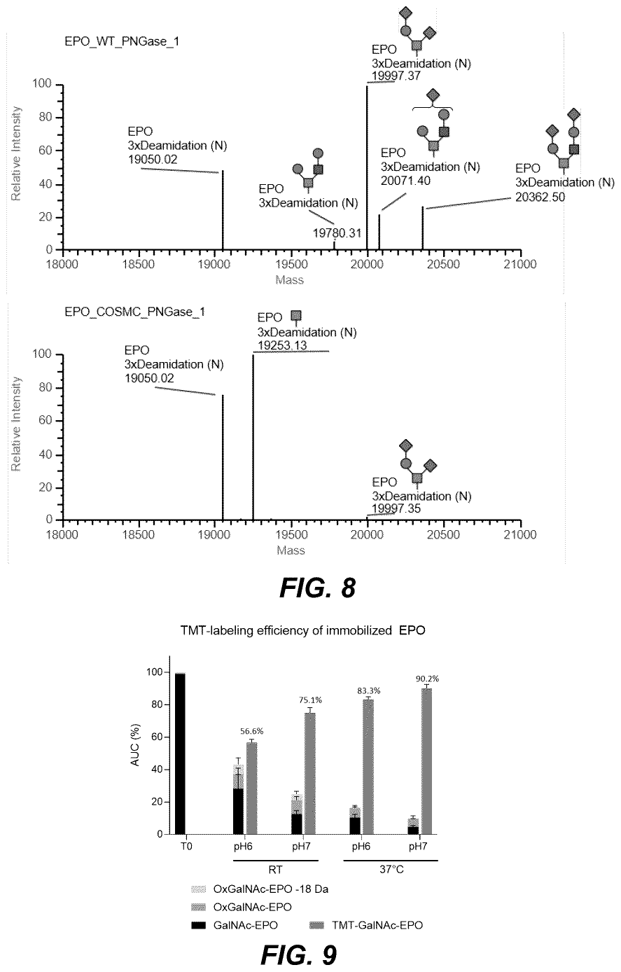

Erythropoietin (EPO) was chosen as internal standard for the Tn-minder methodology, since this glycoprotein has a single O-glycan on a tryptic peptide of 15 amino acids, which was found to be identifiable by LC-MS/MS (Lai et al., 1986; Wang et al., 2019). EPO is a kidney-secreted hormonal protein that circulates in the blood at very low levels (~11 μg/mL in healthy people) (Coles et al 1992) should be virtually absent in (healthy) tissue biopts, which makes it a first suitable internal standard for the set-up. To ensure the homogenous presence of the Tn-antigen, EPO was produced in a SimpleCell version of HEK293S cells (Steentoft et al., 2011). This cell line was developed in-house by knocking-out all four copies of the COSMC gene via Crispr-Cas9 technology. EPO protein with a C-terminal hexa-histidine tag, was produced in WT HEK293S cells as well as in HEK293S COSMC^{KO} cells, and subsequently purified via immobilized metal affinity chromatography (IMAC, Ni²⁺-NTA) followed by size exclusion chromatography (SEC). The purified protein was digested with PNGaseF, to remove the N-glycans that should be present at three different N-glycosylation sites (Lai et al., 1986). PNGaseF thus reduces the macro- and microheterogeneity, which is needed for proper O-glycan characterization via intact protein LC-MS. The de-N-glycosylated and non-reduced EPO samples were analyzed on an LTQ-XL mass spectrometer and MS spectra were deconvoluted with BioPharma Finder (Xtract Protein Deconvolution). The resulting, deconvoluted spectra are shown in FIG. 8. PNGaseF transforms N-glycosylated Asn's to deamidated Asn's (=Asp's). Since EPO has three N-glycan sites, three deamidated Asn residues were expected if N-glycan site occupancy was complete. The masses of the O-glycosylation variants of EPO that were found in WT-produced EPO were consistent with differentially sialylated core 1 and core 2 structures, as expected (FIG. 8, top panel) (Chin et al., 2019; de Haan et al., 2022). The Tn-antigen was virtually homogenous in COSMC^{KO}-produced EPO, further referred to as Tn-EPO (FIG. 8, bottom panel). The relative intensity of each glycovariant in all three replicates of Tn-EPO is listed in Table 1. On average, 60.5% of the Tn-EPO (that will be used as internal) standard contains the Tn-antigen, while 1.6% is represented by the doubly sialylated core-1 glycan. Detection of a low level of doubly sialylated core-1 O-glycosylation on Tn-EPO was not unexpected (FIG. 8, Table 1). Cosmc is a chaperone for the core 1 synthase; in its absence a low level of core 1 synthesis is often still possible. The levels are very low though and may easily go undetected. For example, Yang et al. expressed EPO in a SimpleCell version of CHO cells and did not detect any elongated O-glycans (Yang et al., 2014). Also, other studies on glycosylation of mucins in HEK293 COSMC^{KO} cells did not report extended type O-glycosylation (Nason et al., 2021; Konstantinidi et al., 2022).

TABLE 1
Relative quantification of O-glycosylation
of EPO produced in HEK293S COSMC^{KO} cells.

| | | | Non- |
|---|---|---|---|
| | GalNAc | Sia(2)GalGalNAc | glycosylated |
| Replicate | (% AUC) | (% AUC) | (% AUC) |
| 1 | 60.52 | 1.37 | 38.11 |
| 2 | 60.47 | 1.57 | 37.97 |
| 3 | 60.48 | 1.72 | 37.80 |
| Mean ± SEM | 60.49 | 1.55 | 37.96 |

EPO was produced in COSMC^{KO} cells and analyzed in triplicate via intact protein LC-MS, after N-glycan removal with PNGaseF. The relative intensities (% AUC) of all O-glyco variants were calculated after deconvolution with BioPharma Finder.

### Example 5. Optimization of GaOX-aminoxyTMT™ Conjugation on Solid Phase

The chemoenzymatic workflow (FIG. 1) was optimized in solution and on the peptide level, though the application on a PVDF-coated 96-well plate requires that all chemoenzymatic steps also work well on immobilized, intact glycoproteins. In a pilot experiment on EPO, the TMT-labelling efficiency dropped to ca. 50%, as compared to 92% labelling efficiency obtained in solution on GalNAc-Muc4 in the same reaction conditions (50 mM NaCitrate buffer of pH 6 and overnight incubation at RT) on the same enzyme/substrate ratio (data not shown). To optimize the labelling efficiency on plate, the sodium citrate buffer at pH 6 (optimum for oxime-ligation) was again compared with a sodium phosphate buffer at pH 7 (optimum for GaOX), both at RT as well as 37° C. Higher temperatures are suboptimal for oxygen dissolution, although closer to the optimum temperature of GaOX (35° C.) (Paukner et al., 2015). These four conditions were tested in triplicate on Tn-EPO that was immobilized on the PVDF-plate and incubated with PNGaseF, prior to addition of the GaOX/aminoxyTMT™ mix. After overnight incubation, the reaction mixture was removed from the wells via extensive washing and Tn-EPO was digested with LysC and trypsin, followed by peptide elution with 20% methanol and 0.1% TFA. These eluted fractions were desalted with C18-resin and analyzed via LC-MS/MS. In fact, the conditions for optimal in-solution labelling (pH 6, RT) resulted in the least efficient conjugation on solid phase (56.6%, FIG. 9). Increasing the pH to 7 in combination with an increase in reaction temperature to 37° C. resulted in a mean conjugation efficiency of 90.2%, which is comparable with the efficiency previously obtained in solution.

Next to evaluating TMT-labelling efficiency, an EPO peptide mapping analysis was performed on the same datasets. Such an analysis aligns the identified peptide sequences from the MS2 spectra with the theoretical sequence of EPO. When a mass difference that corresponds with a predetermined PTM is detected between the theoretical sequence and the observed precursor mass of the aligned peptide, it will be reported and, if possible, localized based on the MS2 spectrum. Peptide mapping was performed with BioPharma Finder (Thermo Fisher), and the human glycan database of BioPharma Finder software was included as a source of possible PTMs as well as deamidation of Asn. A peptide sequence coverage of 73.68% was obtained, covering all N-glycosites. The only identified PTM for all tryptic N-glycopeptides was deamidation of Asn, with a reported relative abundance of 100%. No N-glycosylated peptides were identified by BioPharma Finder, indicating efficient N-glycan removal by PNGaseF on the PVDF-membrane, as expected given that the PVDF-well plate sample preparation method has its origins in N-glycome sample preparation and has been in use for this purpose for over 20 years (Callewaert et al., 2001; Jacobs et al., 2009).

### Example 6. Optimization Setup for the Integrated Tn-Miner Methodology

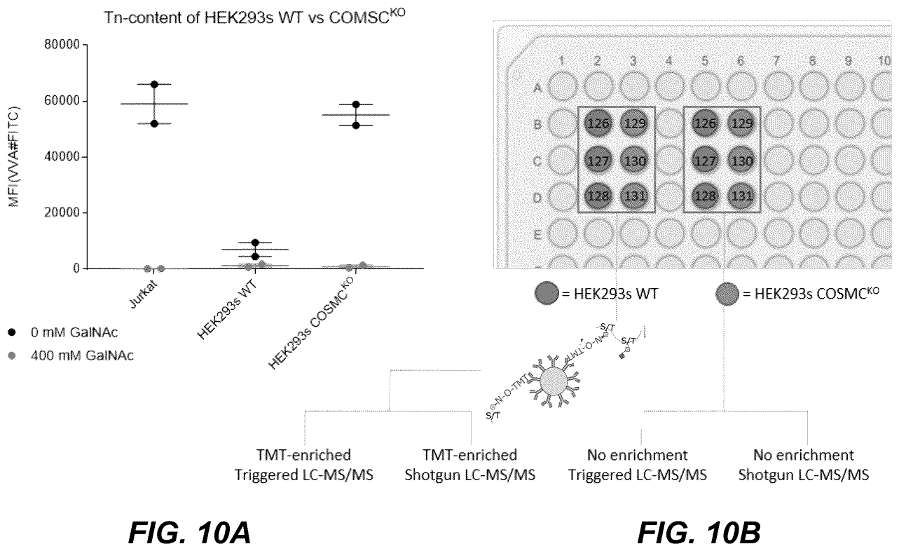

The complete chemoenzymatic workflow as depicted in FIG. 1 and explained in Example 1, was performed for this pilot experiment, although restricted to the left path in order to obtain as much labelled GalNAc-moieties as possible. PM-protein enriched lysates of HEK293S WT and HEK293S COSMC^{KO} cells are analyzed with the chemoenzymatic workflow in a sixplex set-up (FIG. 10B). The Tn-content of the cell surface of both cell lines was assessed via flow cytometry, with Jurkat cells as a positive control (FIG. 10A). An 8-fold increase in MFI obtained from VVA-FITC binding was observed in the COSMC^{KO} HEK293S line, as compared to the WT (FIG. 10A).

For each cell line, ca. 20×10⁶ cells were lysed and their PM was enriched with the Minute™ plasma membrane protein isolation and cell fractionation kit from Invent Biotechnologies. These PM-protein enriched pellets were re-dissolved in denaturing buffer, which contains 8 M ureum, and Tn-EPO internal standard was added to both protein suspensions. The amount of internal standard that was added was calculated in such a way that the final amount of Tn-EPO per well of the PVDF-plate was equal to 1 μmol (≈600 fmol of Tn-glycosylated EPO), 15 μg of PM-enriched protein suspension was loaded on each well. The disulfide bonds were reduced with dithiothreitol (DTT) and free thiols were subsequently blocked by iodacetamide (IAA). The PVDF-coated 96-well plate was loaded as depicted in FIG. 10B. The numbers on this figure depict the reporter masses of the TMT-label that will be added to each well following protein digestion. After elution from the plate, all samples within the same sixplex set-up (squares, FIG. 10B) were combined and dried via vacuum centrifugation. These dried samples were re-dissolved in 5% ACN, 0.5% TFA and purified over C18 resin to remove LysC and trypsin and buffer components. The removal of both proteases is needed to avoid digestion of the antiTMT antibodies in the subsequent TMT-enrichment step. Only one of the pooled sixplex samples was enriched over antiTMT resin (FIG. 10B), the other one was cleaned with C18 resin and dissolved in buffer A (2% ACN, 0.1% TFA) for LC-MS/MS analysis. Both sixplex samples were split in two parts: one was analyzed via the TMT-GalNAc triggered LC-MS/MS workflow, as optimized in Example 3, and the other one was analyzed via shotgun LC-MS/MS with HCD (NCE 40%). Both analyses were performed on the Orbitrap Fusion™ Lumos™ Tribrid™. This experiment resulted in four different datasets, of two different samples, each with a different level of enrichment (FIG. 10B). Each dataset was hence derived from 7.5 μg of loaded PM-enriched proteome per sample, containing ~300 fmol of the Tn-EPO standard (which is ~9 ng).

### Proof of Concept: EPO Internal Standard

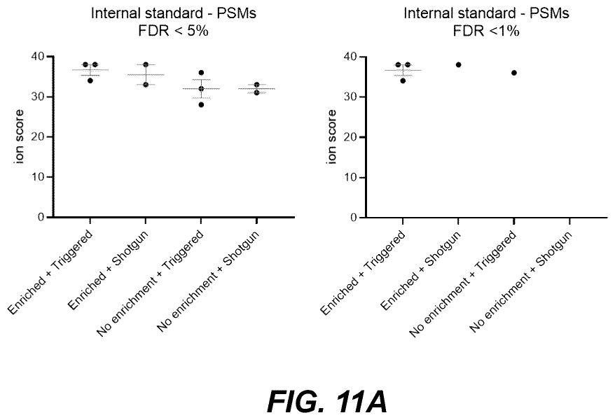

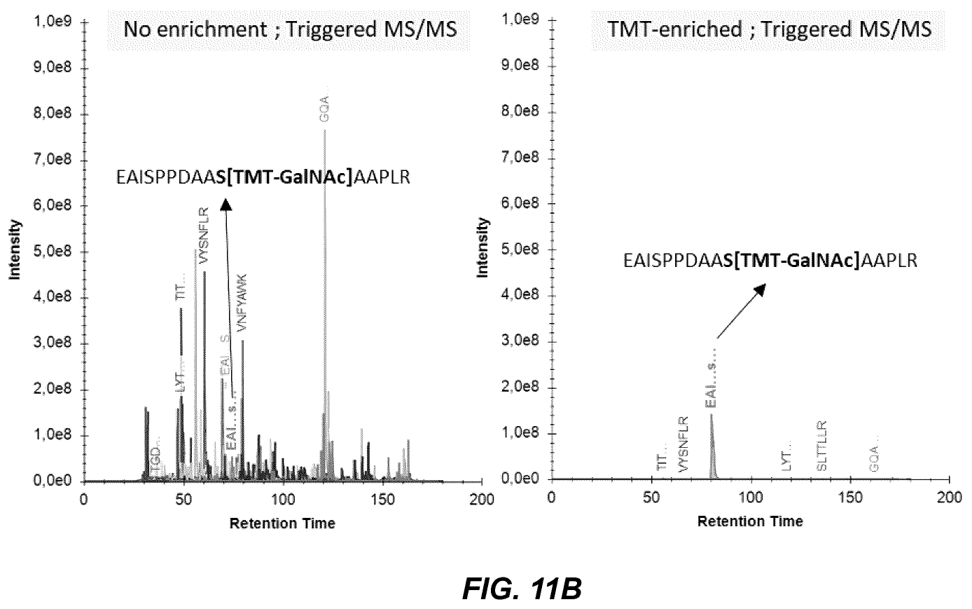

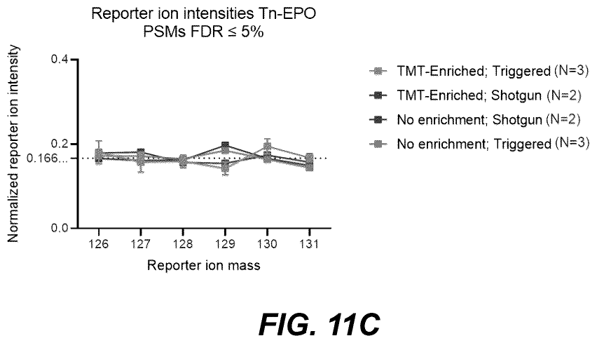

All datasets were analyzed with Proteome Discoverer (PD) by extracting all HCD spectra followed by a search with Mascot against the human proteome database from Swiss-Prot (version of January 2021). The tryptic O-glycopeptide of EPO was identified in all samples with TMT-GalNAc as modification. These TMT-GalNAc-EPO PSMs and their ion scores are depicted in FIG. 11A, where the highest ion scores were observed when TMT-enrichment was combined with the triggered LC-MS/MS method. In this enriched and triggered analysis, all PSMs (three in total) of the O-glycopeptide of EPO scored above the strict ion score threshold for FDR<1%. When either only TMT-enrichment or triggered MS/MS was deployed, only one PSM could be withheld with strict FDR scoring. If no enrichment nor triggered MS/MS was performed, only two PSMs were found with an FDR<5%, which score below the strict ion score threshold for FDR<1% (FIG. 11A). This result shows the value of the double-selective workflow to reliably identify Tn modified peptides in complex proteome backgrounds.

The extracted ion chromatograms (XIC) of all EPO peptides are plotted in FIG. 11B. By comparing the XIC traces of the TMT-enriched with the non-enriched samples, it is clear that the TMT-enrichment was successful. Indeed, the intensities of all EPO peptides, except for the TMT-labelled O-glycopeptide, were drastically decreased after TMT-enrichment, while an increase in ion intensity of the TMT-labelled peptide was observed (FIG. 11B). This increase in intensity can be explained by a reduction in ion suppression due to the removal of the bulk of non-target peptides, resulting in less proton competition within the ESI source.

To assess the well-effect, the reporter ion intensities of the TMT-labelled Tn-glycopeptide of EPO were analyzed. For this, all reporter ions of PSMs with an FDR<5% (i.e., all PSMs depicted in the left panel of FIG. 11A) were taken into account. The reporter ion intensities were normalized by ‘row normalization’ as described by Cologna et al. (2015). For each PSM, the ‘row sum’ of the reporter ions was calculated, which is the sum of the intensities of all different reporter ions for that specific PSM. Each of the individual reporter ion intensities was then divided by the ‘row sum’, resulting in a number that can be interpreted as ‘fraction of the total intensity’. It is expected that each well of the sixplex set-up to have an equal amount of Tn-EPO, represented by equally intense reporter ion peaks. After row normalization, this expected value should therefore be one-. sixth. As shown in FIG. 11C, all reporter ions are indeed aligned at this expected value.

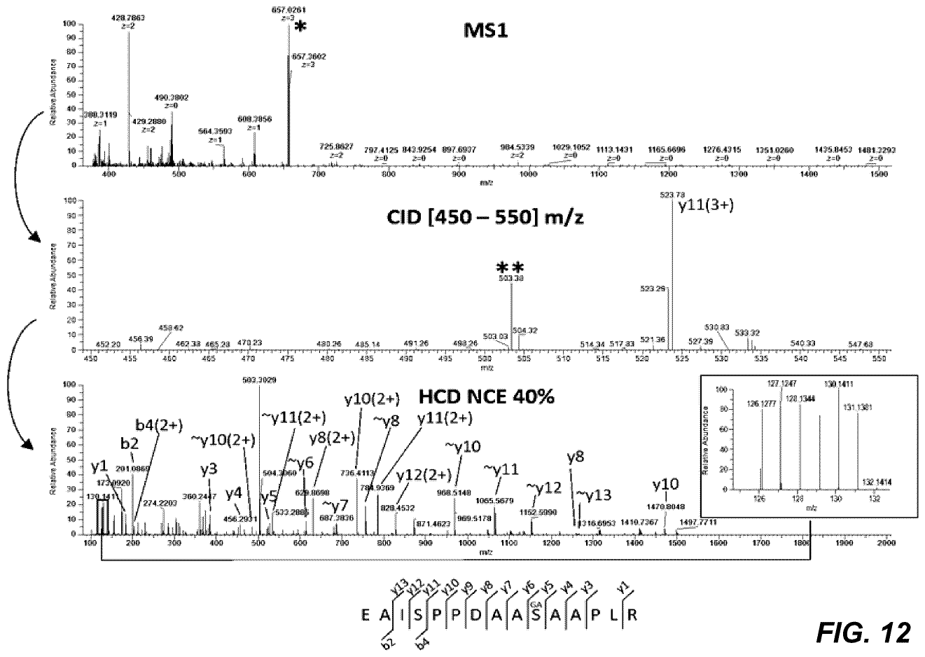

An example of the triggered workflow with subsequent quantification is depicted in FIG. 12. The MS1 spectrum containing the triple charged, tryptic TMT-GalNAc-EPO peptide (asterisk, FIG. 12) is shown in the upper panel. This precursor ion is selected for fragmentation by CID in the ion trap, with an m/z range of [450-550] m/z. A clear 503.3 m/z ion (two asterisks, FIG. 12) is detected with a relative intensity above the 20% threshold, resulting in a triggered HCD spectrum with NCE of 40%. The latter spectrum contains all six reporter ions (zoom), which allows relative quantification of this Tn-epitope (FIG. 13).

Overall Peptide Yield: TMT-Enrichment Vs. No Enrichment

Right before LC-MS/MS analysis, the total peptide concentration of both the non-enriched and TMT-enriched sample was measured via UV-absorbance on the Lunatic UV/Vis absorbance spectrometer (Trinean, Unchained labs). A total peptide yield of 6.4 μg (0.18 μg/μL) for the non-enriched samples was obtained, which is ca. 7.1% of the total amount of input material (ca. 90 μg=6 wells of 15 μg of protein). This loss of material can be explained by the combination of incomplete binding to PVDF and loss of material during washing steps, plate elution and C18 clean-up. Based on the relative quantification of EPO, this loss of material is reproducible for every well, otherwise a difference in reporter ion ratios would be observed (FIG. 11C). The Lunatic instrument was however unable to determine the peptide concentration of the TMT-enriched samples, which was probably below its reported detection limit of 0.02 μg/μL. The enriched sample was dissolved in 35 μL of buffer A, and a decision was made to inject the maximal injection volume of 15 μL for each LC-MS/MS analysis.

For the non-enriched samples, 2 μg was injected per LC-MS/MS run. The maximal peak intensity of the total ion current (TIC) can be considered as a rough estimate for the number of ions that entered the mass spectrometer. The maximal intensity of the TIC within a window of 80 to 120 min was extracted, to assure that only peptide derived peaks are included. This intensity was on average 12.4 times higher in the non-enriched samples compared to the enriched samples (FIG. 13). This means that roughly 12 times less material was injected for the enriched samples (2 μg/12=0.17 μg) (FIG. 13). If this value is recalculated to the original concentration, 0.011 μg/μL is obtained, which is indeed below the detection limit of the Lunatic instrument. A decrease in TIC is, of course, expected after enrichment, since the bulk of non-target peptides should be eliminated prior to LC-MS/MS analysis. It should be noted that the signal for the TMT-labelled O-glycopeptide from EPO actually doubled in intensity after enrichment (FIG. 11B). This observation states that the actual signal of target peptides is not decreased by a factor 12 but might even be increased due to the reduction in ion suppression. This result also indicates that the recovery of TMT-labelled Tn-glycopeptides in the eluate of the purification step must be high, although it cannot be exactly quantified using these data. At the same time, it may be rather unlikely that 1/12^{th} of the peptides would be O-glycopeptides, and hence there may be scope for a repeated anti-TMT-tag purification to further deplete the background peptidome, which could boost the detectability of the target TMT-labelled Tn-glycopeptides. Although, this comes with a cost of (again) material loss, inherent to aspecific peptide binding to the plastic of the column and to the agarose matrix toward which the antiTMT-antibodies are coupled.

### Identification of Tn-glycopeptides in HEK293S WT and HEK293S COSMC^{KO} Lysates

A numerical overview of all HCD derived PSMs obtained from the four different samples, with all EPO related PSMs excluded, is given in Table 2. In the first column, the total amount of PSMs (ranked first by Mascot) are depicted at different FDR filters. The number of PSMs in the second column are filtered based on the combined presence of reporter ions of 129, 130 and 131 m/z, since these were used for the labelling of COSMC^{KO} cell lysates and more Tn-antigens were expected in these samples compared to the WT. In the third column, the total number of PSMs is given where Mascot has assigned the TMT-GalNAc modification. The last column represents PSMs that contain both the 129, 130 and 131 m/z reporter ions and are identified with TMT-GalNAc as PTM. The latter PSMs should, in theory, be the most trustworthy, considering that only reporter ions are expected when a TMT-GalNAc is present, due to the specificity of the chemoenzymatic workflow, especially when the TMT-GalNAc dependent trigger method is deployed.

TABLE 2
Numerical overview of the PSMs obtained
from the four different datasets.

| | | | |
|---|---|---|---|
| | contains | | Contains 129, 130 |
| | 129, 130 and | | and 131 m/z |
| #PSMs | 131 m/z | TMT-GalNAc | and TMT-GalNAc |

TMT-Enriched + Triggered MS/MS

| | | | | |
|---|---|---|---|---|
| total | 204 | 16 | 27 | 4 |
| FDR <5% | 31 | 1 | 0 | 0 |
| FDR <1% | 22 | 1 | 0 | 0 |

TMT-Enriched + Shotgun

| | | | | |
|---|---|---|---|---|
| total | 1127 | 117 | 115 | 7 |
| FDR <5% | 226 | 34 | 0 | 0 |
| FDR <1% | 144 | 21 | 0 | 0 |

No enrichment + Triggered MS/MS

| | | | | |
|---|---|---|---|---|
| total | 3106 | 465 | 58 | 6 |
| FDR <5% | 1300 | 209 | 0 | 0 |
| FDR <1% | 965 | 139 | 0 | 0 |

No enrichment + Shotgun

| | | | | |
|---|---|---|---|---|
| total | 20438 | 4955 | 306 | 46 |
| FDR <5% | 10581 | 2577 | 0 | 0 |
| FDR <1% | 8294 | 1914 | 0 | 0 |

The total number of PSMs were extracted for every dataset, with different FDR filters. Two extra filters were applied: (1) the presence of the combination of all reporter ions specific for the wells with COSMC^{KO} lysates (129, 130 and 131 m/z). (2) Identification of TMT-GalNAc as variable modification. The right column depicts a combination of filters 1 and 2.

Most strikingly for all four samples was the discrepancy between the number of PSMs with reporter ions in the three COSMC^{KO} specific channels and the number of PSMs that were annotated with TMT-GalNAc. Both groups were expected to largely overlap (number in the right column), as discussed above. For each PSM in the enriched samples with reporter ions in the COSMC^{KO} specific channels, the ion fill times (i.e., the time needed to acquire enough ions in the orbitrap to obtain a proper MS/MS spectrum) reached the maximum of 120 ms. In other words, too few ions (<7.5E4 ions) were present for the generation of MS/MS spectra of sufficient quality, which is also translated to the very low ion scores of these PSMs. It is very likely that the spectra with reporter ions are actually derived from TMT-labelled Tn-glycopeptides, but were wrongly annotated by Mascot due to the poor spectral quality. When only a few fragment ion peaks are available for spectral matching, Mascot might assign the spectrum to a wrong peptide sequence that coincidently matches with these few peaks without the presence of TMT-GalNAc as modification. For the non-enriched samples, ca. 25% of all PSMs that contained reporter ions in the COSMC^{KO} specific channels had ion fill times below 120 ms. Further analysis of these spectra will be performed to investigate the hypothesis.

Although only four, low scoring PSMs were obtained in the TMT-enriched+triggered MS/MS dataset that contain reporter ions in the COSMC^{KO} specific channels as well as TMT-GalNAc as modification (Table 2, right column), it should be noted that two of them were already annotated as O-glycoprotein in literature. The Golgi integral membrane protein 4 (GOLIM4) is annotated to the Golgi apparatus as well as to the endosomes and the PM, according to UniProt. Two SimpleCell based studies (Steentoft et al., 2013; Campos et al, 2015) have identified the same Tn-glycosylation site (Ser405) as shown herein. The other identified Tn-glycopeptide was identified as Glucosidase 2 subunit beta (GLU2B), where a Tn-antigen was detected on the peptide spanning S282 to K301. The same peptide was identified as an O-GalNAc-containing peptide by the two same SimpleCell studies (Steentoft et al., 2013; Campos et al, 2015). GLU2B was also picked up in the non-enriched, triggered file. However, GLU2B is annotated as an ER-specific protein and not as PM-associated protein by UniProt.

### Example 7. Implementation of the POC Data for Applying the Integrated Tn-Miner Methodology

In general, the complete chemoenzymatic workflow on solid phase with subsequent clean-up and enrichment steps comes with the cost of extensive material losses, resulting in very low amounts of TMT-GalNAc labelled ions that can be analyzed via LC-MS/MS. Due to these low ion numbers, it seems that MS/MS spectra of sufficient quality could not yet be generated. However, taking into account the sample prep recovery of 7%, it was (roughly) calculated that ca. 126 fmol of Tn-EPO was present before TMT-enrichment. Assuming that 90% of this Tn-EPO is labelled with TMT and that the material loss during TMT-enrichment is rather low, it could be estimated that for each experiment ca. 100 fmol TMT-Tn-EPO was injected for LC-MS/MS analysis. This 100 fmol TMT-Tn-EPO resulted in an ion peak intensity of ca. 2×10⁸ (FIG. 11B), which was translated in high quality spectra with reliable identification (FDR<1%) and performant quantification, when TMT-enrichment and the triggered LC-MS/MS workflow were deployed. As can be seen on the TIC chromatogram of this sample (FIG. 13, lower left panel), the bulk of ions reaches only 0.5×10⁸ to 1×10⁸ as absolute signal intensity. This back-of-the-envelope calculation suggests that the absolute amount of TMT-Tn-labelled peptides should increase at least fourfold to obtain high quality spectra of the most prevalent (Sia)Tn-glycopeptide epitopes in the PM-proteome. It should be noted that good immunotherapy targets should not be exceedingly low in abundance on tumor cell surfaces, as tumor killing by effector cells requires multivalent immune cell triggering. Hence, reliably profiling the top few 100 sites is what is desired to achieve.

The low abundancy of TMT-Tn-glycopeptides might be solved by increasing the number of wells per sample, in combination with an optimization of peptide elution of the plate. The group of H. Steen described efficient peptide elution from this type of PVDF-coated plates using 40% ACN:0.1% formic acid (FA) (Berger et al., 2015), while 20% MeOH: 0.1% TFA was used. Changing TFA to FA might improve the peptide elution, since TFA can increase the hydrophobicity of peptides via ion pairing, an effect that is less pronounced for FA (Shibue et al., 2005). However, increasing the hydrophobicity of the elution buffer might result in coelution of polyvinylpyrrolidone 360 (PVP360), which is used to block non-specific binding spots of the wells. If PVP360 would be co-eluted, it will partially compete with the peptides for binding to the C18 resin in the subsequent clean-up step, again possibly resulting in peptide loss. The MS-tern blot protocol does not need blocking agents (Berger et al., 2015), allowing to increase the concentration of the organic solvent for peptide elution without major problems. As reviewed by the group of S. Flitsch, immobilizing a substrate in enzymatic reactions often comes with the cost of a decrease in enzyme-substrate binding rate, which results in lower enzymatic efficiencies. This is often solved by increasing the enzyme concentration or enzyme diffusion (Halling et al., 2005). In the method described herein, the amount of LysC and trypsin may be increased to increase the overall peptide yield, as it has yet to be assessed whether proteolysis is complete for the PVDF-immobilized proteins. If Tn-EPO is zoomed in on, the chemoenzymatic workflow is efficient in conjugating aminoxyTMT™ to the Tn-antigen on solid phase and the subsequent TMT-enrichment in combination with triggered MS/MS results in high ion scores and performant relative quantification. In addition, protein binding and peptide elution for each PVDF-coated well of the two sixplex set-ups was reproducible. The TMT-labelling efficiency was equal on each well; otherwise differences in reporter ion quantification would have been detected. One might argue whether the combination of TMT-enrichment and triggered MS/MS is beneficial for the identification of new, possible Tn-glycopeptide epitopes, since much more PSMs were obtained when both filters were omitted. Based on the data, both enrichment steps will however be needed if an increase in peptide material can be achieved by optimizing the factors discussed above. If the total amount of peptides increases, the TMT-labelled Tn-glycopeptides will become more prevalent in absolute numbers, although their relative amount compared to the bulk of non-target peptides will remain the same. From the EPO data, it can be concluded that enrichment and MS/MS triggering was able to specifically isolate the TMT-labelled O-glycopeptide of EPO with performant identification and quantification. This level of selectivity will allow to remove the bulk of non-target peptides, while identifying the TMT-labelled Tn-glycopeptide that are hidden in this haystack of peptides.

It should be noted that some tumor types are described to express the EPO receptor in combination with EPO, which induces proliferation of the cancer cells (Debeljak et al., 2014). A more universal internal standard could easily be manufactured, using the SimpleCell HEK293S cells described herein. For instance, EPO-variants with point mutations in the Tn-carrying tryptic peptide can be designed and produced inspired by sequence variants in related non-human primate species, such that distinguishing it from endogenous human EPO is possible.

### Example 8. Evaluation of Tn-Miner Workflow Applying a Higher Amounts of Starting Material

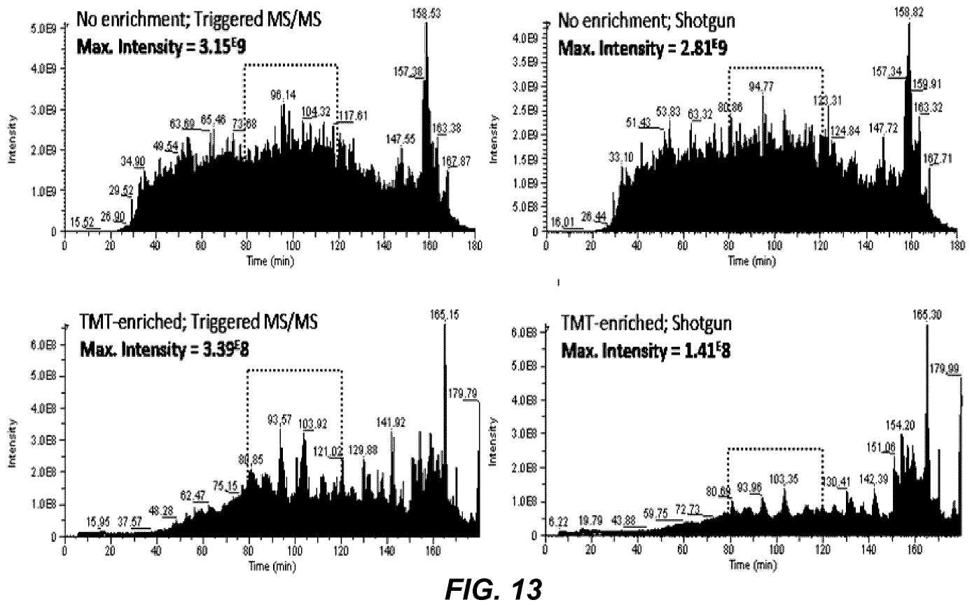

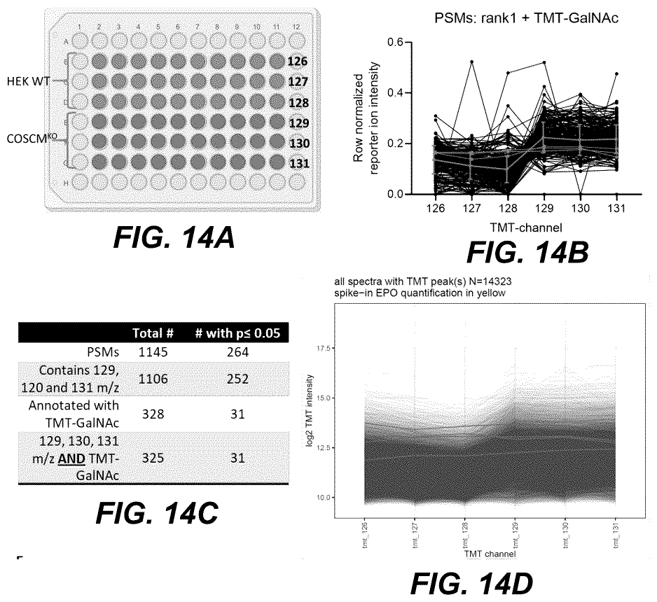

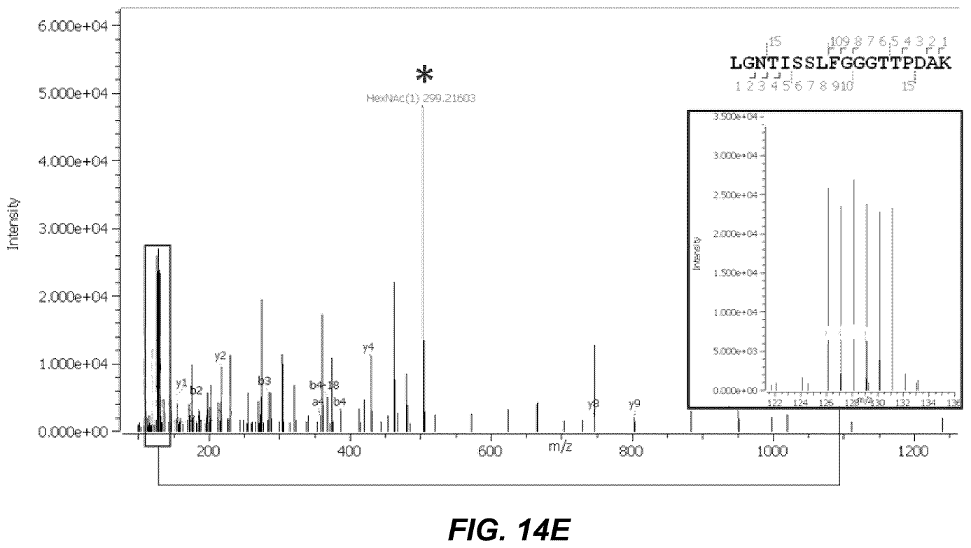

The Tn-Miner workflow was evaluated in an additional experiment on both HEK293s WT cells and HEK293s COSMC^{KO} cells. For this evaluation test, the PM-enriched fraction of each cell line was loaded on 30 different wells of a PVDF-coated porous 96-well plate as to compensate for the loss of material observed in previous testing (FIG. 14A). Each well was loaded with 10 μg of the corresponding cell line, with addition of 100 fmol of Tn-EPO as internal standard. The WT lysates were labeled with TMT126, 127 and 128, while the COSMC^{KO} lysates were labeled with TMT129, 130 and 131. After completion of on-plate chemoenzymatic workflow and protein digestion, the peptides were eluted from the plate, combined into one sample and desalted over C18 resin. The desalted sample was loaded on 200 μL of antiTMT-beads for TMT-enrichment. After elution from the beads, the sample was dried and redissolved in 30 μL of a 2% ACN; 0.1% TFA buffer. Of this sample, 10 μL was injected for LC-MS/MS analysis, deploying the triggered MS/MS workflow as described in FIG. 4. The raw data was analyzed with Proteome Discoverer, with Mascot as search engine.

As depicted in Table 3, the mass spectrometer was triggered 15,201 times to acquire an HCD spectrum. The vast majority of these spectra (12,701 or 83.6%) contained all three COSMC^{KO}-specific reporter ions, meaning that ca. 84% of all acquired HCD spectra were originating from target TMT-Tn-glycopeptides. Of these 15,201 HCD spectra, 1145 PSMs (rank 1) were obtained by Mascot, of which 328 were annotated to be modified with TMT-GalNAc (FIG. 14C).

Of these 1145, 264 PSMs had a p-value≤5%. The majority of spectra from these PSMs (1106) had reporter ions in the COSMC^{KO} specific channels, of which 31 had a p-value≤5%. Mascot was able to annotate 328 spectra with a peptide containing the TMT-GalNAc modification, of which 325 had all the reporter ions corresponding to the COSMC^{KO} channels.

When applying statistics, only 328 of these had a p-value≤0.05, corresponding to 16 unique glycosites (UGS). This low identification rate can be explained by the nature of HCD fragmentation, where the most labile bonds, being the O-glycosidic bond and reporter ion bonds in this case, have the highest chance to break upon fragmentation. This often results in scarce spectra that are heavily dominated by the TMT-GalNAc oxonium ion and reporter ions and contain only limited y- and b-ions originating from the amino acid backbone (FIG. 14E). Another reason for the low identification rate is the likely presence of other O-glycans on the same glycopeptide, resulting in complex fragmentation spectra.

FIG. 14B shows all row normalized reporter ions intensities from the PSMs that were annotated with TMT-GalNAc (rank 1). The internal standard, TMT-Tn-EPO, is depicted in red, the mean intensity with SEM is depicted in yellow.

FIG. 14D shows all log 2 transformed reporter ion intensities from the spectra that had at least one TMT-reporter ion in the spectrum. All reporter ion intensities from the same spectrum are connected with a black line. The reporter ion intensity of the internal standard, TMT-Tn-EPO, is depicted in yellow. As expected, an equal amount of TMT-Tn-EPO over all samples was observed, while the other PSMs show an overall increase in the COSMC^{KO} channels.

Although only 31 PSMs (corresponding to 16 UGS) were identified with high confidence, it can be assumed that the vast majority of HCD spectra are most likely originating from (Sia)Tn-antigen containing glycopeptides. This assumption is corroborated by the presence of the TMT-GalNAc-signature ion in the CID spectrum that resulted in a triggered HCD spectrum, containing the expected TMT-reporter ions. In addition, these peptides were still present within the sample after thorough TMT-enrichment, again proving the presence of the TMT-label. All these acquired m/z values can be confidently used for a second PRM-based analysis with ETD, EThcD, sceHCD or EAD fragmentation, in order to localize and identify these peptides with higher certainty.

TABLE 3
Numerical summary of CID-triggered HCD spectra from
the proof of concept experiments in Examples 8 and 9.

| | |
|---|---|
| Example 8 | Example 9 |

#HCD triggered spectra

| | |
|---|---|
| 15,201 | 15,125 |

#Spectra with ALL COSMCKO reporters

| | |
|---|---|
| 12,701 | 12,526 |
| 83.6% | 82.8% |

#PSMs

| | |
|---|---|
| 1145 | 1693 |

#PSMs - TMT-GalNAc

| | |
|---|---|
| 328 | 204 |

#PSMs - TMT-GalNAc - p ≤ 0.05

| | |
|---|---|
| 31 | 31 |

#UGS - TMT-GalNAc - p ≤ 0.05

| | |
|---|---|
| 16 | 12 |

### Example 9. Repetition of the Setup of Example 8, Extended with PRM Analysis

The experiment described in Example 8 was repeated with the following adaptations: (I) no TFA was added for elution of the peptides from the PVDF membrane; (II) instead of 200 μL antiTMT-resin, 450 μL was used; (III) the enriched TMT-Tn-Glycopeptides were resuspended in 30 μL 2% ACN prior to LC-MS/MS analysis, without the addition of TFA. Acids were avoided during the Tn-Miner protocol to limit acid catalysed hydrolysis of the formed oxime bond between GalNAc and the TMT label.

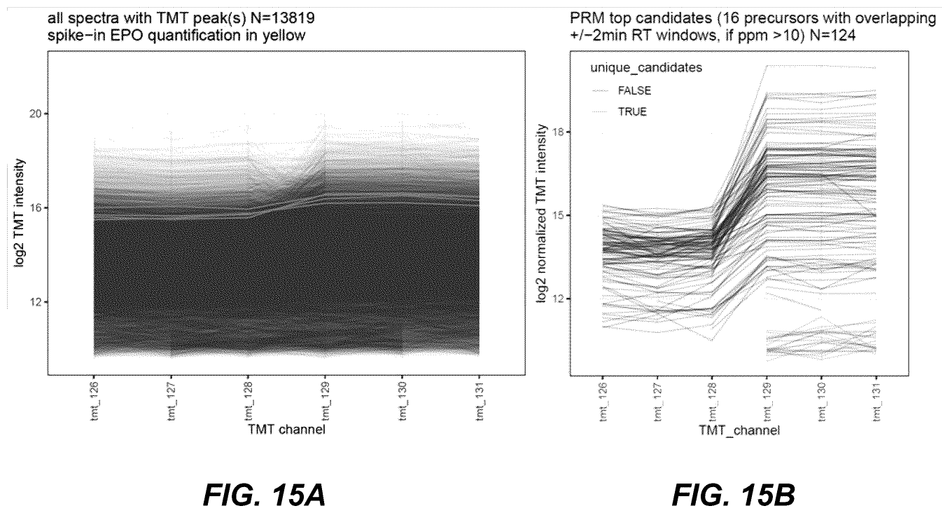

In a first ‘profiling run’, the same LC-MS/MS analysis was performed as described in Example 8 and as shown in FIG. 4. The raw data of this run was analyzed with Proteome Discoverer, with Mascot as search engine. The comparison between the output of Example 8 and 9 is shown in Table 3. From this comparison it can be concluded that the number of generated spectra and number of PSMs and UGS is reproducible over the two, independent experiments. The log 2 transformed reporter ion intensities of all spectra that had at least one reporter ion, are shown in FIG. 15A. Compared to the intensities obtained from the experiment of Example 8 (FIGS. 14B & 14D), an overall increase in intensity could be observed for de data in Example 9, compared to Example 8. This is the positive consequence of the avoidance of acids during the sample prep as well as the increase in anti-TMT resin volume. The difference in reporter ion intensities of the HEK293s WT compared to HEK293 COSMC^{KO} is less pronounced as in Example 8, which is most likely due to the overall increase in signal. The intensities of the WT specific reporter ions are now also above the limit of quantitation of the mass spectrometer, giving a more realistic view on the amount of (Sia)Tn-antigen for the HEK293s WT cell line, which is in fact an immortalized cells showing a lot of cancer related characteristics (Stepanenko & Dmitrenko, 2015).

This initial profiling run results in a list of triggered spectra (i.e., containing the TMT-GalNAc signature ion), of which the elution time, precursor m/z value and relative quantification is known based on the reporter ion intensities of the TMT label. The information obtained from this profiling run is needed as input for the mass spectrometer to perform a second parallel reaction monitoring (PRM) run. In a PRM type experiment, the mass spectrometer is programmed to exclusively focus on a predefined list of m/z values (i.e., peptide ions of interest), within predefined time windows of the elution profile. In this way, both time and space of the mass spectrometer are exclusively used for the analysis of peptides of interest, resulting in higher number of collected ions within the mass detector and more time for suboptimal, chemical fragmentation methods such as ETD and EAD. These fragmentation methods result in cleavage of the amino acid backbone, rather than the O-glycosidic bond, which leads to higher identification rates and proper O-glycan localization.

The selection of most interesting m/z values for PRM analysis was performed via an in-house developed R-script that prioritizes the m/z values that are exclusively represented in the COSMC^{KO} reporter ion channels, supplemented with m/z values that are most significantly differentially expressed between the WT and COSMC^{KO} channels (FIG. 15B). The maximal number of m/z values that can be isolated per cycle of the mass spectrometer is also calculated by the script in such a way that two to three points across the chromatographic peak are analyzed for each eluting peptide of interest. For this experiment, a maximum of 16 precursors for each four minute window was calculated, resulting in a selection of 124 m/z values of interest. The ion reporter intensities of these candidates are shown in FIG. 15B.

The PRM analysis of the 124 top candidates was performed with ETD fragmentation, followed by analysis with Byonic. This search engine is dedicated for glycoproteomics searches and is one of the most ETD-compatible search engines on the market (Bern et al., 2012). Byonic scores of 200 or more are considered to be trustworthy. The summary of the analysis is given in Table 4. Ca. 3000 ETD spectra were obtained from the 124 targeted precursor masses, resulting in 1027 PSMs of which 951 were annotated as being modified with TMT-GalNAc. Of these PSMs, 277 had a score over 200, representing 15 UGS with localized glycosite.

TABLE 4
Numerical summary of the spectra generated
by PRM analysis with ETD fragmentation.

| | | #PSMs | #PSMs | #UGS |
|---|---|---|---|---|
| #ETD | | TMT- | TMT-GalNAc - | TMT-GalNAc - |
| Spectra | #PSMs | GalNAc | score ≥ 200 | score ≥ 200 |
| 2,994 | 1027 | 951 | 277 | 15 |

It should be noted that the focus was on the differentially expressed (Sia)Tn-antigens between HEK293s WT cells and HEK293s COSMC^{KO} cells to mimic a ‘healthy vs. tumor tissue’ situation, while HEK293s WT is in fact an immortalized cell line with a lot of cancer related characteristics (Stepanenko & Dmitrenko, 2015). Due to this set-up, the majority of interesting (Sia)Tn-antigens were filtered out during the differential expression filtering, resulting in a low amount of targets of interest, which all had a low absolute abundance within the sample. In a follow-up experiment, different cancer cell lines will be compared within one multiplex experiment, focussing on precursor masses that are present in all cell lines, ranked based on reporter ion intensity. Here, a higher number of precursor masses is expected that will be included in the PRM list (ca. 1000 precursors per PRM analysis), with high absolute abundance within the sample.

### Example 10. Evaluation of the IMPa Glycoprotease to Decrease Glycopeptide Heterogeneity Prior to LC-MS/MS Analysis

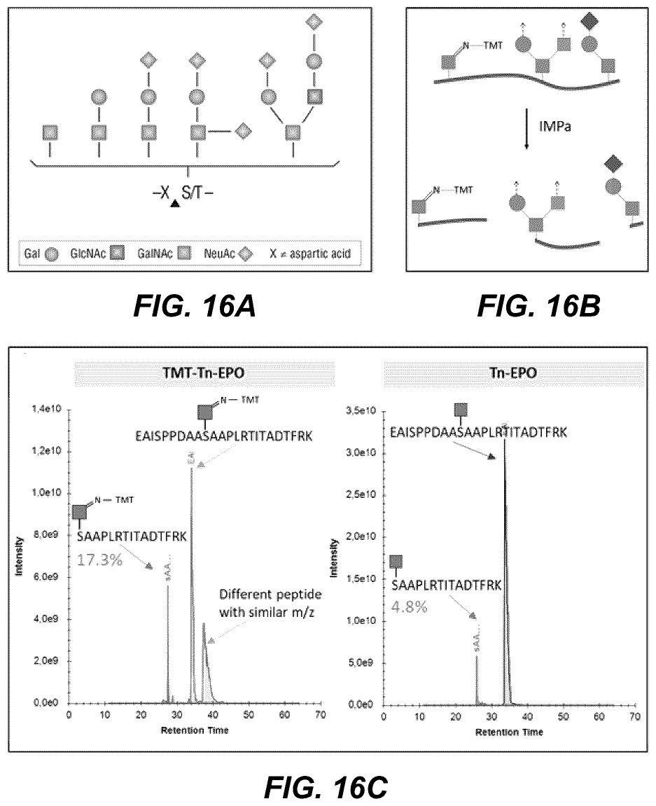

One of the major issues for proper peptide identification is the likely presence of heterogeneous non-target glycans on (Sia)Tn-antigen containing peptides. This results in a complex, heterogeneous glycopeptide mixture. If search engines like Byonic are programmed to consider this huge amount of possible glycan combinations as variable modification, the search space expands significantly, demanding substantial computational power. Also, this huge search space results in an enormous amount of possible annotations, often leading to wrong identification. To avoid this issue, the implementation of a broad-spectrum O-glycoprotease, IMPa, was evaluated. This IMPa protease, also known as Immunomodulating protease (IMPa) from Pseudomonas aeruginosa, is described to hydrolyze proteins and peptides N-terminally of O-glycosylated Ser/Thr residues, with very limited bias to the amino acid backbone (Riley & Bertozzi, 2022; Vainauskas, et al. 2022) (FIG. 16A). Digestion of the TMT-Tn-glycopeptides with this protease, should hence result in peptides with a single, N-terminal glycan. If these peptides are eluted from the PVDF plate and enriched for the TMT-labeled peptides, the sample should only contain glycopeptides with a single TMT-Tn-antigen on their N-terminus, reducing the complexity tremendously (FIG. 16B).

In a first experiment, the activity of IMPa on a TMT-Tn-glycoprotein was evaluated, which is a non-natural substrate for this protease. For this, Tn-EPO was immobilized on two wells of a PVDF coated 96-well plate, followed by N-glycan removal with PNGaseF. For one of the wells, the Tn-antigen was labelled with aminoxyTMT by incubation with GaOX/aminoxyTMT. The Tn antigen in another well was not labelled and served as a positive control, since the native Tn-antigen is a known substrate for IMPa (Riley & Bertozzi, 2022; Vainauskas, et al. 2022). Both wells were incubated with IMPa for 3h, followed by an overnight incubation with LysC. All peptides were eluted and subsequently analyzed on LC-MS/MS. Approximately 17% of the TMT-Tn-EPO was hydrolyzed N-terminally of Ser-Tn-TMT, while only approximately 5% was hydrolyzed in the case of Tn-EPO (FIG. 16C). It has been proven that the TMT-Tn-antigen can serve as substrate for the IMPa glycoprotease, possibly with even higher efficiency compared to the non-modified Tn-antigen. However, it should be noted that these percentages are likely an underestimation of the real hydrolysis efficiency, due to the limited ionization efficiency of the short, IMPa/LysC digested peptides, compared to the longer and the highly ionizable IMPa miss-cleaved form.

This step thus provides for a valid perhaps more suitable alternative of digestion of the glycoproteins prior to analysis through mass spectrometry, aiding to identify a higher number of isolated (Sia)Tn-antigen peptides.

### Materials and Methods

### Sialylation of Galactose to Protect Against Oxidation by GaOx.

For sialylation, 500 μmol GalGalNAc-Muc4 and 500 μmol GalNAc-Muc4 were mixed with 50 nmol CMP-Neu5Ac (BioSynth, Cat. No MC04391), 0.45 μg ST3Gal1 (R&D systems, Cat. No. 6905-GT-020) and 0.74 μg ST3Gal3 (R&D systems, Cat. No. 10554-GT) in a total reaction volume of 250 μL 25 mM TrisHCl pH 7.5. Incubation was performed at 37° C. while shaking. Aliquots were taken at certain timepoints, which were snap frozen to stop the reaction. For the oxidation assays, 500 μmol of fucosylated/sialylated glycopeptide (still in the sialylation/fucosylation buffer with enzymes) was desalted over C18 spin column (ThermoFischer, Cat. No. 89870) as described by the manufacturer. The dried peptide pools were redissolved in 50 μL 50 mM NaPᵢ buffer pH 7 with 2U GaOX and incubated ON at 21° C. while shaking.

### TMT-Labelling of the Pilot Peptide Pool.

The O-GalNAc SpikeMix™ (JPT, Cat. No. SPT-PTM-POOL-Glyco-OGalNAc-1), containing 100 synthetic Tn-glycopeptides (10 μmol/peptide) was dissolved in 93 μL MilliQ grade water and split in three samples of 30 μL each. Every part was mixed with 28 μg of a different aminoxTMT™ (126, 127 and 128) and 8 U GaOX in a total volume of 250 μL of a 50 mM NaCitrate buffer pH 6. The samples were incubated at room temperature overnight, while shaking and were subsequently dried via vacuum centrifugation, redissolved in 50 μL of MilliQ grade water and dried again (to boost the labelling efficiency, as described by the manufacturer). The samples were redissolved in 10% acetone and incubated for 10 min at room temperature while shaking, to quench aminoxy group. The samples were dried via vacuum centrifugation and each part was redissolved in 30 μL of loading solvent A (0.1% TFA in water/ACN (98:2, v/v)) after which the samples were combined by adding 20 μL and 10 μL of the TMT127 and TMT128 sample, respectively, to the 30 μL of TMT126 sample. The excess of quenched TMT was removed by gel filtration over Sephadex G-10 resin (Cytiva, Cat. No. 17001002). In short, the mixed sample is loaded per 10 μL on a well of a porous 96-well plate filled with the G-10 resin, with subsequent centrifugation for 10 sec at 750 g. The flowthrough is collected in a receiver plate and loading solvent A (0.1% TFA in water/ACN (98:2, v/v)) is added to the eluens to final volume of 75 μL for LC-MS/MS analysis.

### LC-MS/MS for Trigger Optimization and HCD Vs EThcD Comparison.

For each LC-MS/MS analysis, 10 μL of the labelled glycopeptide pool was injected on an Ultimate 3000 RSLCnano system in-line connected to a Fusion Lumos mass spectrometer (Thermo). Trapping was performed at 20 μl/min for 2 min in loading solvent A on a 5 mm trapping column (Pepmap, 300 μm internal diameter (I.D.), 5 μm beads, C18, Thermo). The peptides were separated on a generation 2 110 cm prototype column (μPAC, Thermo), kept at a constant temperature of 50° C. Peptides were eluted by a non-linear gradient starting from 4% MS solvent B (0.1% FA in Acetonitrile) reaching 26.4% MS solvent B in 45 min and 44% MS solvent B in 55 min and 56% MS solvent B in 60 min starting at a flowrate of 600 nl/min for 5 minutes, and completing the run at a flow rate of 300 nl/min, followed by a 5-minute wash at 56% MS solvent B and eventually 25 min re-equilibration with 96% MS solvent A (0.1% FA in water). The mass spectrometer was operated in Top Speed mode with a cycle time of 3s. Full-scan MS spectra (375-1500 m/z) were acquired at a resolution of 120,000 in the Orbitrap analyzer after accumulation to a target AGC value of 100,000 with a maximum injection time of 50 ms. The precursor ions were filtered for charge states (3-7), dynamic exclusion (60 s; +/−10 ppm window, n=2) and intensity (minimal intensity of 5E3).

For the HCD NCE optimization, the precursor ions were selected in the quadrupole with an isolation window of 1.2 Da and accumulated to an AGC target of 1E4 or a maximum injection time of 80 ms and activated using CID fragmentation (30% NCE). The fragments were analyzed in the Ion Trap Analyzer at rapid scan rate over a small mass range (200-550 m/z). The most intense precursors were selected in the quadrupole according to the cycle time of 3s in total. The precursors leading to a fragment ions of 503.3 m/z and 252.2 m/z (tolerance 0.3 m/z) above a product ion threshold of 5, were re-isolated in the quadrupole with an isolation window of 1.2 m/z, an AGC target of 7.5E4 and a max injection time of 120 ms. An HCD fragmentation was performed on these selected precursors with NCE 40, 50 or 60, measuring the fragments in the orbitrap with a resolution of 30000, with the first mass set to 100.

For the single 503.3 m/z trigger method with relative threshold above 20%, the method parameters were adapted as described in example 2. In short, only the Ion Trap mass range changed to 450-550 m/z and the precursors leading to a fragment ion of 503.3 m/z (tolerance 0.3 m/z) above a product ion threshold of 20, were re-isolated in the quadrupole with the same settings as described for the dual trigger method.

The MS/MS settings of the EThcD based method with optimized, single trigger were as follows: The mass spectrometer was operated in Top Speed mode with a cycle time of 3s. Full-scan MS spectra (375-1500 m/z) were acquired at a resolution of 120,000 in the Orbitrap analyzer after accumulation to a target AGC value of 100,000 with a maximum injection time of 50 ms. The precursor ions were filtered for charge states (3-7), dynamic exclusion (60 s; +/−10 ppm window, n=2) and intensity (minimal intensity of 5E3). The precursor ions were selected in the quadrupole with an isolation window of 1.2 Da and accumulated to an AGC target of 1E4 or a maximum injection time of 80 ms and activated using CID fragmentation (30% NCE). The fragments were analyzed in the Ion Trap Analyzer at rapid scan rate over a small mass range (450-550 m/z). The most intense precursors were selected in the quadrupole according to the cycle time of 3s in total. The precursors leading to a fragment ion of 503.3 m/z (tolerance 0.3 m/z) above a product ion threshold of 20, were re-isolated in the quadrupole with an isolation window of 1.2 m/z, an AGC target of 5E4 and a max injection time of 200 ms. An ETD fragmentation was performed on these selected precursors with calibrated charge dependent ETD parameters, measuring the fragments in the iontrap at normal scan rate, with an automatically set mass range, followed by an HCD supplemental activation (NCE 30), accumulating 3 microscans.

### Generation of a HEK293S COSMC^{KO} Strain.

Ribonucleoprotein (RNP) complexes were generated by combining 40 μM Cas9/GFP protein (produced and purified by the VIB Protein Core Facility) in Nucleofector™ solution (P3 Primary Cell 4D-Nucleofector™ X Kit S, Lonza, Cat. No. V4XP-3032) with 80 μM COSMC-specific gRNA (UUUUUGAAGGGUGUGAUGCU (e.g., SEQ ID NO:5)) in MilliQ grade, nuclease free water. One million HEK293S cells, grown in a 50:50 mixture of FreeStyle™ 293 expression medium (Gibco™, Cat. No. 12338018) and EX-CELL® 293 serum-free medium (Merck, Cat. No. 14571C), were pelleted and resuspended in 20 μL Nucleofector™ solution. This suspension was added to 4 μL of the RNP solution and transferred to a nucleofector cuvette. After electroporation by the 4D-X core unit (Lonza, Cat. No. AAF-1003B), 80 μL of prewarmed medium was added to the cells, which were placed in a humidified incubator to recover (37° C., 5% CO₂). The nucleofected cell culture was expanded by culturing in DMEM/F-12 (Gibco, Cat. No. 11320033) supplemented with 10% FCS (Gibco, Cat. No. 10270-106) and 33% preconditioned medium. Single cells were obtained by cell sorting, 7 days post nucleofection. Before and after expansion, sequencing of the gDNA targeted site was performed, followed by analysis with the ICE tool of Synthego to assess knock-out efficiency. Clones with a knock-out efficiency >95% were selected for phenotype screening by VVA-FITC staining and flow cytometry analysis (data not shown).

### VVA-FITC Staining and Flow Cytometry Analysis.

After collection, cells were immediately put on ice and washed once with ice-cold PBS (Lonza, Cat. No. BE17-516F). Each cell line was resuspended in ice-cold PBS and loaded in the wells of a V-bottom 96-well plate to obtain 500 000 cells/well. After centrifugation at 300 g for 5 min, PBS was removed from the plate and cells were resuspended in 200 μL staining buffer containing 5 μg VVA-FITC (EY Labs, Cat. No. F-4601-5) and 0.2 L eBioscience™ Fixable Viability Dye eFluor™ 780 (ThermoFisher, Cat. No. 65-0865-18) in PBS. For the non-specific binding control, VVA-FITC was preincubated with 400 mM D-GalNAc in PBS. For both VVA-FITC and the viability dye, single stain controls were taken along to optimize the compensation matrix on the LSR II flow cytometer (BD Lifesciences). After resuspension in staining buffer, the plate was put on RT for 15 min in the dark, followed by two washing steps with ice-cold PBS. Cells were resuspended in 100 μL PBS and loaded on the LSR II flow cytometer. The cells were subsequently gated for non-debris, single cells and living cells. The flow cytometry results were analyzed using FlowJo™ v10.8 Software (BD Life Sciences).

### Production of EPO and LC-MS Analysis.

Recombinant EPO protein production was performed via transient transfection of HEK293S or HEK293S COSMC^{KO} cells with the pcDNA3.3-TOPO-TA expression vector encoding the human EPO gene coupled to a C-terminal 6×His-tag. Recombinant EPO was purified from the supernatant three days post transfection via IMAC on a Ni²⁺-NTA packed column followed by size exclusion on a HiLoad 16/600 Superdex 75 μg column. Prior to LC-MS, 20 μg EPO was diluted in a final volume of 100 μL, 50 mM triethylammonium bicarbonate (TEAB) followed by addition of 52 mU PNGaseF and 2 h incubation at 37° C., while shaking. The sample was acidified to 1% TFA and 2 μg was injected on the HPLC system. Intact LC-MS and data analysis was performed as described by Schepens et al. (2021), but with a Zorbax Poroshell 300SB-C8 LC column (5 μm, 300 Å, 1×75 mm ID×L; Agilent Technologies) as only adaptation.

### Optimization of TMT-Labelling on Solid Phase.

Fresh urea buffer (8 M urea, 3.2 mM EDTA in 360 mM Tris·acetate, pH 8.6) was added to 225 μg Tn-EPO to a final volume of 750 μL, after which Tn-EPO was reduced and alkylated by 20 min incubation at 50° C. with 5 mM dithiothreitol followed by incubation with 10 mM iodoacetamide for 15 min at RT in the dark. Two PVDF-coated 96-well plates (Merck, Cat. No. MSIPN4510) were mounted on a vacuum manifold, and the wells were activated with 300 μL MeOH, followed by three washes with 300 μL MilliQ grade water and one wash with fresh urea buffer. The sample was divided over the activated wells of the two plates (15 μg per well), followed by centrifugation for 2 min at 1000 g with collection of the flow through in a receiver plate. The flow through was re-applied two times. After washing the wells three times with MilliQ grade water, aspecific binding spots were blocked by 1 h incubation with 1% polyvinylpyrrolidone 360 (PVP360) at room temperature. The PVP was removed and the wells were washed 3 times with MilliQ grade water and once with 50 mM NaPᵢ ph7 or 50 mM NaCitrate pH6. The plate was removed from the vacuum manifold and 50 μL of a GaOX-TMT mastermix was applied to all wells (160 U/mL GaOX and 400 μg/mL aminoxyTMT™ in 50 mM NaPᵢ pH7 or 50 mM NaCitrate pH6). After ON incubation at 37° C. or RT in a humidified chamber, the GaOX-TMT mix was removed via the vacuum manifold and the wells were washed three times with MilliQ grade water. Digestion was performed by incubation with 0.5 μg LysC (NEB, Cat. No. P8109S) in 50 mM TEAB for 2 h at 37° C., followed by the addition of 0.5 μg Trypsin (Promega, Cat. No. V5117), and ON incubation at 37° C. The peptides were eluted via centrifugation (1000 g, 2 min) in a collector 96-well plate and the wells were washed three times with 50 μL 20% MeOH, 0.1% TFA. Each of the fractions was combined with the corresponding elutions, which were subsequently dried via vacuum centrifugation. The dried fractions were redissolved in 5% acetonitrile, 0.5% TFA and cleaned over a C18 spin column (Thermo Scientific, Cat. No. 89870) as described by the manufacturer. The dried peptide pools were stored at −20° C. until further use.

### The Chemoenzymatic Workflow on HEK293S Cells.

First, PM-proteome enrichment was performed on 20E6 HEK293S cells and an equal amount of HEK293S COSMC^{KO} with the Minute™ Plasma Membrane Protein Isolation and Cell Fractionation Kit (Invent Biotechnologies, Cat. No. SM-005) as described by the manufacturer. The PM-enriched pellets were redissolved in 400 μL fresh urea and protein concentration was determined on a Lunatic instrument (Unchained Lab). Tn-EPO was spiked in both samples in such a way that the final amount of Tn-EPO per well was equal to 1 μmol, taken into account that 15 μg of PM-enriched protein suspension should be loaded on each well. The samples were reduced, alkylated and subsequently loaded on the activated wells (15 μg/well) of a PVDF-coated 96-well plates as described above. After protein loading, the aspecific binding spots were blocked by one hour incubation with 100 μL 1% PVP 360 at room temperature. The blocking solution was removed and the wells were washed two times with MilliQ grade water (300 μL/wash) and once with 50 μL 10 mM TrisAcetate pH 8.3. Then, 50 μL of the PNGaseF reaction mix (1 mU/μL PNGaseF in 10 mM TrisAcetate pH 8.3) was added to each well, followed by 2 h incubation at 37° C. in a humidified box. This reaction mixture was removed, followed by two washing steps with MilliQ grade water and a single wash with 50 μL 5 mM ammonium acetate pH 5. After addition of 50 μL sialidase reaction mix (8 mU/μL α2-3,6,8,9 Neuraminidase in 5 mM ammonium acetate pH 5), the plate was incubated for 90 min at 37° C. in a humidified box. Both PNGaseF and α2-3,6,8,9 Neuraminidase were produced in-house by the Protein Core Facility of VIB. The sialidase mix was removed, followed by three washing steps with MilliQ grade water and once with 50 μL 25 mM TrisHCl pH 7.5. Protection of galactoses was performed by addition of 50 μL ST3Gal1/3 mix to each well (0.2 mM CMP-Neu5Ac, 1.8 ng/μL ST3Gal1 and 3 ng/μL ST3Gal3 in 25 mM TrisHCl pH 7.5). CMP-Neu5Ac was purchased from BioSynth (Cat. No MC04391) and both ST3Gal1 and ST3Gal3 were purchased from R&D systems (Cat. No. 6905-GT-020 and Cat. No. 10554-GT, respectively). After 2 h incubation at 37° C. in a humidified box, the reaction mixture was removed and the wells were washed two times with MiliQ grade water and once with 50 μL 50 mM NaPᵢ pH 7. Then, 8U GaOX (in-house production) and 20 μg aminoxyTMT™ (Thermo Fischer, Cat. No. 90401) were added to each well in a total volume of 50 μL 50 mM NaPᵢ pH 7, followed by overnight incubation at 37° C. in a humidified box. The wells were washed three times with MilliQ grade water and once with 50 μL 50 mM TEAB. On plate digestion was performed by addition of 50 μL of the LysC (NEB, Cat. No. P8109S) mastermix (10 ng/μL LysC in 50 mM TEAB). After 4 h incubation at 37° C. in a humidified box, 0.5 μg trypsin (Promega, Cat. No. V5117) was added per well and the incubation was continued overnight. The peptides were eluted via centrifugation (1000 g, 2 min) in a collector 96-well plate and the wells were washed three times with 50 μL 20% MeOH, 0.1% TFA. All eluted fractions and wash fractions within the same sixplex experiment were combined and subsequently dried via vacuum centrifugation. The dried fractions were redissolved in 5% acetonitrile, 0.5% TFA and desalted over a C18 spin column (Thermo Scientific, Cat. No. 89870) as described by the manufacturer. TMT-labelled peptides were enriched with 150 μL Immobilized Anti-TMT™ Antibody Resin (i.e., 300 μL slurry) (ThermoFischer, Cat. No. 90076), as described by the manufacturer. This enrichment step was performed one day prior to LC-MS/MS analysis.

### LC-MS/MS

Purified peptides were re-dissolved in 35 μl of loading solvent A (0.1% TFA in water/ACN (98:2 v/v)), unless stated otherwise. For the enriched sample, 15 μl was injected for each analysis. For the non-enriched samples, the peptide concentration was determined on a Lunatic instrument (Unchained Lab) and 2 μg was injected. For the proof of concept experiments in Example 8 and 9, 10 μL was loaded for LC-MS/MS analysis on an Ultimate 3000 RSLCnano system in-line connected to a Fusion Lumos mass spectrometer (Thermo). Trapping was performed at 20 μl/min for 2 min in loading solvent A on a 5 mm trapping column (Pepmap, 300 μm internal diameter (I.D.), 5 μm beads, C18, Thermo). The peptides were separated on a generation 2 110 cm prototype column (μPAC, Thermo), kept at a constant temperature of 50° C. Peptides were eluted by a non-linear gradient starting from 2% MS solvent B (0.1% FA in Acetonitrile) reaching 26.4% MS solvent B in 137 min and 44% MS solvent B in 150 min and 56% MS solvent B in 160 min starting at a flowrate of 500 μl/min for 5 minutes, and completing the run at a flow rate of 300 nl/min, followed by a 5-minute wash at 56% MS solvent B and eventually 15 min re-equilibration with 96% MS solvent A (0.1% FA in water).

For the TMT-GalNAc triggered analysis, the mass spectrometer was operated in Top Speed mode with a cycle time of 3s. Full-scan MS spectra (375-1500 m/z) were acquired at a resolution of 120,000 in the Orbitrap analyzer after accumulation to a target AGC value of 100,000 with a maximum injection time of 50 ms. The precursor ions were filtered for charge states (3-7), dynamic exclusion (60 s; +/−10 ppm window, n=2) and intensity (minimal intensity of 5E3). The precursor ions were selected in the quadrupole with an isolation window of 1.2 Da and accumulated to an AGC target of 1E4 or a maximum injection time of 80 ms and activated using CID fragmentation (30% NCE). The fragments were analyzed in the Ion Trap Analyzer at rapid scan rate over a small mass range (450-550 m/z). The most intense precursors were selected in the quadrupole according to the cycle time of 3s in total. The precursor leading to a fragment ion of 503.3 m/z (tolerance 0.3 m/z) above a product ion threshold of 20, were re-isolated in the quadrupole with an isolation window of 1.2 m/z, an AGC target of 7.5E4 and a max injection time of 120 ms. An HCD fragmentation was performed on these selected precursors with NCE 40, measuring the fragments in the orbitrap with a resolution of 30000, with the first mass set to 100.

For the shotgun analysis, the mass spectrometer was operated in Top Speed mode with a cycle time of 3 s. Full-scan MS spectra (375-1500 m/z) were acquired at a resolution of 120,000 in the Orbitrap analyzer after accumulation to a target AGC value of 100,000 with a maximum injection time of 50 ms. The precursor ions were filtered for charge states (3-7), dynamic exclusion (60 s; +/−10 ppm window, n=2) and intensity (minimal intensity of 5E3). The precursor ions were selected in the quadrupole with an isolation window of 1.2 Da and accumulated to an AGC target of 1E4 or a maximum injection time of 120 ms and activated using HCD with NCE of 40. Fragments were measured in the orbitrap with a resolution of 30000 and a defined first mass of 100 m/z.

For the PRM analysis, the precursor ions that matched the m/z values of the predefined PRM list (m/z window+/−10 ppm, RT window+/−4 min), were selected in the quadrupole with an isolation window of 0.7 Da and accumulated to an AGC target of 250,000 with a maximum injection time of 250 ms and activated using ETD with calibrated charge dependent ETD parameters. Fragments were measured in the orbitrap with a resolution of 30,000 and an automatic mass range. The mass spectrometer was programmed to perform cycles of 16 scans, each time followed by an MS1 scan. MS1 scans were acquired at a resolution of 30000 (375-1500 m/z) in the Orbitrap analyzer after an optimized accumulation target value with a maximum injection time of 45 ms.

### IMPa Digestion

Tn-EPO (20 μg) was diluted to a final volume of 100 μL in fresh urea-buffer (8 M urea, 3.2 mM EDTA in 360 mM Tris·acetate, pH 8.6). Tn-EPO was subsequently reduced and alkylated by 20 min incubation at 50° C. with 5 mM dithiothreitol followed by incubation with 10 mM iodoacetamide for 15 min at RT in the dark. A PVDF-coated 96-well plates (Merck, Cat. No. MSIPN4510) was mounted on a vacuum manifold, and two wells were activated with 300 μL MeOH, followed by three washes with 300 μL MilliQ grade water and one wash with fresh urea buffer. The sample was divided over the activated wells of the two plates (10 μg per well), followed by centrifugation for 2 min at 1000 g with collection of the flow through in a receiver plate. The flow through was re-applied two times. After washing the wells three times with MilliQ grade water, aspecific binding spots were blocked by 1 h incubation with 1% polyvinylpyrrolidone 360 (PVP360) at room temperature. The PVP was removed and the wells were washed 3 times with MilliQ grade water and once with 10 mM TrisAcetate pH 8.3. Then, 50 μL of the PNGaseF reaction mix (1 mU/μL PNGaseF in 10 mM TrisAcetate pH 8.3) was added to each well, followed by 2 h incubation at 37° C. in a humidified box. This reaction mixture was removed, followed by two washing steps with MilliQ grade water and a single wash with 50 μL 20 mM Tris-HCl pH 8.0. Then, 1U the IMPa (NEB, Cat. No. P0761S) in 50 μL 20 mM Tris-HCl pH 8.0 was added to one well, while the other was incubated in buffer only. After 3 h incubation at 37° C. in a humidified box, 0.5 μg LysC (NEB, Cat. No. P8109S) was added an incubation continued ON at 37° C. Peptides were eluted and collected by centrifugation at 1000 g for 2 min, followed by two wash steps with 20% MeOH in MilliQ grade water. The eluted samples were dried via vacuum centrifugation and redissolved in MilliQ grade water prior to LC-MS/MS analysis. AUC analysis of all expected EPO derived peptides was performed with Skyline. For the IMPa efficiency, the AUC of the glycosylated IMPa/LysC resulting peptide was divided by the AUC of the total pool of (TMT-)Tn-peptides.

### REFERENCES

Avigad, G., Amaral, D., Asensio, C. & Horecker, B. L. The d-Galactose Oxidase of Polyporus circinatus. J. Biol. Chem. 237, 2736-2743 (1962).
Baba, T. et al. Localization of Multiple O-Linked Glycans Exhibited in Isomeric Glycopeptides by Hot Electron Capture Dissociation. J. Proteome Res. 21, 2462-2471 (2022).
Berger, S. T. et al. MStern Blotting-High Throughput Polyvinylidene Fluoride (PVDF) Membrane-Based Proteomic Sample Preparation for 96-Well Plates. Mol. Cell. Proteomics MCP 14, 2814-2823 (2015).
Bern, M., Kil, Y. J. & Becker, C. Byonic: Advanced Peptide and Protein Identification Software. Curr. Protoc. Bioinforma. 40, 13.20.1-13.20.14 (2012).
Callewaert, N., Geysens, S., Molemans, F. & Contreras, R. Ultrasensitive profiling and sequencing of N-linked oligosaccharides using standard DNA-sequencing equipment. Glycobiology 11, 275-281 (2001).
Campos, D. et al. Probing the O-glycoproteome of gastric cancer cell lines for biomarker discovery. Mol. Cell. Proteomics MCP 14, 1616-1629 (2015).
Chin, C. L. et al. A human expression system based on HEK293 for the stable production of recombinant erythropoietin. Sci. Rep. 9, 16768 (2019).
Coles, G. A. et al. Estimation of erythropoietin secretion rate in normal and uremic subjects. Am. J. Physiol.-Ren. Physiol. 263, F939-F944 (1992).
Cologna, S. M. et al. An Efficient Approach to Evaluate Reporter Ion Behavior from MALDI-MS/MS Data for Quantification Studies using Isobaric Tags. J. Proteome Res. 14, 4169-4178 (2015).
de Haan, N. et al. In-Depth Profiling of O-Glycan Isomers in Human Cells Using C18 Nanoliquid Chromatography-Mass Spectrometry and Glycogenomics. Anal. Chem. 94, 4343-4351 (2022).
Debeljak, N., Solár, P. & Sytkowski, A. J. Erythropoietin and Cancer: The Unintended Consequences of Anemia Correction. Front. Immunol. 5, (2014).
Griffin, T. J. et al. iTRAQ reagent-based quantitative proteomic analysis on a linear ion trap mass spectrometer. J. Proteome Res. 6, 4200-4209 (2007).
Gupta, R., Matta, K. L. & Neelamegham, S. A systematic analysis of acceptor specificity and reaction kinetics of five human a (2,3) sialyltransferases: Product inhibition studies illustrates reaction mechanism for ST3Gal-I. Biochem. Biophys. Res. Commun. 469, 606-612 (2016).
Halling, P. J., Ulijn, R. V. & Flitsch, S. L. Understanding enzyme action on immobilised substrates. Curr. Opin. Biotechnol. 16, 385-392 (2005).
Jacobs, P. P., Geysens, S., Vervecken, W., Contreras, R. & Callewaert, N. Engineering complex-type N-glycosylation in Pichia pastoris using GlycoSwitch technology. Nat. Protoc. 4, 58-70 (2009).
Jeanneau, C. et al. Structure-Function Analysis of the Human Sialyltransferase ST3Gal I: ROLE OF N-GLYCOSYLATION AND A NOVEL CONSERVED SIALYLMOTIF*. J. Biol. Chem. 279, 13461-13468 (2004).
Kitagawa, H. & Paulson, J. C. Differential expression of five sialyltransferase genes in human tissues. J. Biol. Chem. 269, 17872-17878 (1994).
Konstantinidi, A. et al. Exploring the glycosylation of mucins by use of O-glycodomain reporters recombinantly expressed in glycoengineered HEK293 cells. J. Biol. Chem. 298, 101784 (2022).
Lai, P. H., Everett, R., Wang, F. F., Arakawa, T. & Goldwasser, E. Structural characterization of human erythropoietin. J. Biol. Chem. 261, 3116-3121 (1986).
Nason, R. et al. Display of the human mucinome with defined O-glycans by gene engineered cells. Nat. Commun. 12, 4070 (2021).
Ortiz-Soto, M. E., Reising, S., Schlosser, A. & Seibel, J. Structural and functional role of disulphide bonds and substrate binding residues of the human beta-galactoside alpha-2,3-sialyltransferase 1 (hST3Gal1). Sci. Rep. 9, 17993 (2019).
Paukner, R., Staudigl, P., Choosri, W., Haltrich, D. & Leitner, C. Expression, purification, and characterization of galactose oxidase of Fusarium sambucinum in E. coli. Protein Expr. Purif. 108, 73-79 (2015).
Riley, N. M. & Bertozzi, C. R. Deciphering O-glycoprotease substrate preferences with O-Pair Search†. Mol. Omics 18, 908-922 (2022).
Schepens, B. et al. An affinity-enhanced, broadly neutralizing heavy chain-only antibody protects against SARS-CoV-2 infection in animal models. Sci. Transl. Med. 13, eabi7826 (2021).
Shibue, M., Mant, C. T. & Hodges, R. S. Effect of anionic ion-pairing reagent hydrophobicity on selectivity of peptide separations by reversed-phase liquid chromatography. J. Chromatogr. A 1080, 68-75 (2005).
Steentoft, C. et al. Mining the O-glycoproteome using zinc-finger nuclease-glycoengineered SimpleCell lines. Nat. Methods 8, 977-982 (2011).
Steentoft, C. et al. Precision mapping of the human O-GalNAc glycoproteome through SimpleCell technology. EMBO J. 32, 1478-1488 (2013).
Stepanenko, A. A. & Dmitrenko, V. V. HEK293 in cell biology and cancer research: phenotype, karyotype, tumorigenicity, and stress-induced genome-phenotype evolution. Gene 569, 182-190 (2015).
Vainauskas, S. et al. A Broad-Specificity O-Glycoprotease That Enables Improved Analysis of Glycoproteins and Glycopeptides Containing Intact Complex O-Glycans. Anal. Chem. 94, 1060-1069 (2022).
Wang, Q. et al. Characterization of intact glycopeptides reveals the impact of culture media on site-specific glycosylation of EPO-Fc fusion protein generated by CHO-GS cells. Biotechnol. Bioeng. 116, 2303-2315 (2019).
Want, E. J., Cravatt, B. F. & Siuzdak, G. The expanding role of mass spectrometry in metabolite profiling and characterization. Chembiochem Eur. J. Chem. Biol. 6, 1941-1951 (2005).
Whittaker, J. W. The radical chemistry of galactose oxidase. Arch. Biochem. Biophys. 433, 227-239 (2005).
Yang, Z. et al. The GalNAc-type O-Glycoproteome of CHO Cells Characterized by the SimpleCell Strategy. Mol. Cell. Proteomics MCP 13, 3224-3235 (2014).
Yang, W., Ao, M., Song, A., Xu, Y., Sokoll, L., and Zhang, H. Mass Spectrometric Mapping of glycoproteins Modified by Tn-Antigen Using Solid-Phase Capture and Enzymatic Release. Anal. Chem. 92, 9230-9238 (2020).
You, X., Yao, Y., Mao, J., Qin, H., Liang, X., Wang, L., and Ye, M. Chemoenzymatic Approach for the Proteomics Analysis of Mucin-Type Core-1 O-Glycosylation in Human Serum. Analytical Chemistry 90 (21), 12714-12722 (2018).
Zheng, J., Xiao, H., and Wu, R. Specific Identification of Glycoproteins Bearing the Tn Antigen in Human Cells. Angew. Chem. Int. Ed. 56, 7107-7111 (2017).

1. A method for isolating a labelled Tn-antigen-peptide from a glycoprotein sample, comprising the steps of:
a. enzymatically treating a glycoprotein sample to remove the N-glycans present on the glycoproteins,
b. sialylating the O-glycans on the glycoprotein using a sialyltransferase specific for Galactose of Gal-β1,3-GalNAc in core 1 or core 2 type glycans, and a sialyltransferase specific for end-standing Galactose within polyLacNAc, and a sialyltransferase-substrate, for protecting the free galactoses against oxidation in step c.,
c. oxidizing the O-linked GalNac and Gal residues using Galactose oxidase (GaOx) and simultaneously label these oxidized glycans,
and optionally:
d. digesting the glycoprotein into peptides,
e. enriching the labelled Tn-peptides by conjugation on a label-specific binding support.

2. The method according to claim 1, wherein the sialyltransferase enzymes in step b. are β-galactoside α2-3 sialyltransferase 1 (ST3Gal1) and one or more enzymes selected of the group of ST3 β-galactoside α-2,3-sialyltransferase 3, 4, and 5 (ST3Gal3, ST3GAL4, ST3GAL5).

3. The method according to claim 1, wherein after step a., before step b., a treatment to hydrolyze the sialyl-residues from STn-antigens is performed as to enrich for labelled Tn-peptides in step e., which derive from Tn-antigens and STn-antigens; optionally wherein the treatment to hydrolyze the sialyl-residues from STn-antigens is performed using a sialidase, a neuraminidase or trifluoroacetic acid (TFA).

4. The method according to claim 1, wherein the glycoprotein sample:
is obtained from a cell or tissue culture, optionally wherein the sample has undergone lysis and was enriched for the plasmamembrane components; or
is obtained from a biological or clinical sample isolated from a subject, optionally wherein the sample has undergone lysis and was enriched for the plasmamembrane components; or
wherein the sample has undergone lysis and was enriched for the plasmamembrane components.

5. The method according to claim 1, wherein the sample in step a. is immobilized on a solid phase, optionally wherein the solid phase is PVDF coated surface.

6. The method according to claim 1, wherein (i) the labelling in step c. is performed using aminoxy-tandem mass tag (TMT) as labelling reagent, (ii) the label-specific binding support comprises anti-TMT resin, or (iii) the labelling in step c. is performed using aminoxy-tandem mass tag (TMT) as labelling reagent and the label-specific binding support comprises anti-TMT resin.

7. The method according to claim 6, wherein the labelling is performed using several TMT-labelling reagents in a multiplex format.

8. The method according to claim 1, wherein the optional digestion of the glycoproteins in step d. is performed by treatment with LysC and trypsin, or with IMPa and LysC.

9. A liquid chromatography-tandem mass spectrometry method to map labelled Tn-antigen-peptides, comprising the steps of:
a. subjecting a labelled(S) Tn-antigen-peptide-containing sample to an MS1 precursor scan,
b. subjecting the sample of step a. to a MS2 collision-induced dissociation (CID) fragmentation within a narrow m/z range and applying a trigger for reisolation of a precursor ion at m/z corresponding to the Tn-antigen-peptide label using a threshold relative ion intensity of at least 20%,
c. subjecting the reisolated precursor ions of step b. to Higher-energy Collisional Dissociation (HCD) fragmentation or a derivative fragmentation method thereof, with implementation of elevated normalized collision energy (NCE) on a high accuracy mass analyzer, for releasing of reporter ions and allowing relative quantification and identification of the (S)Tn-antigen-peptides.

10. The method according to claim 9, wherein the labelled(S) Tn-antigen peptides are enriched and/or carry a TMT label.

11. The method according to claim 9, wherein a charge filter of ≥2+ or ≥3+ is applied in step a.

12. The method according to claim 9, wherein;
(i) the narrow range in step b. is an m/z range of [450-550], or
(ii) a trigger for ions of 503.3 m/z is applied for reisolation of the precursor ion, or
(iii) the HCD in step c. is applied at an NCE of at least 40%, or
(iv) any combination of (i), (ii) and (iii).

13. The method according to claim 9, further comprising the step d., in parallel to step c., of including the reisolated precursor m/z in the trigger list for parallel reaction monitoring (PRM) analysis during a second LC-MS/MS run of the sample, wherein said second run is with Electron-Transfer Dissociation (ETD), Electron-Transfer/Higher-Energy Collision Dissociation (EThcD) or Electron Activated Dissociation (EAD) fragmentation, for localizing the TMT-Tn-antigen position on the peptide.

14. An integrated method to identify O-linked glycosylation sites of Tn antigen on cell surface glycoproteins, comprising the steps of:
a. isolating labelled Tn-antigen-peptides from a glycoprotein sample according to the method claim 1, and optionally releasing the labelled Tn-antigen-peptides from the enriched support, and
b. subjecting the sample of a. to an LC-MS/MS method for mapping the labelled Tn-antigen peptides.

15. The method according to claim 14, further comprising a step c. and d., which are a repetition of step a. and b., respectively, wherein the enriched Tn-antigen-peptides in step c. derive from Tn-antigen and STn-antigen according to the method of claim 3, and further comprising the step e., comparing the Tn-antigen-peptides identified in step d. and step b. as to identify the STn-antigen-peptides as those identified in step d., but not in step b.

16. A kit comprising ST3Gal1, ST3Gal3, and GaOx enzymes, and a sialyltransferase substrate for use in the method of claim 1.

17. The kit according to claim 16, further comprising (i) an enzyme to hydrolyze sialyl-residues (ii) an enzyme to remove N-glycans from glycoprotein Asn-residues, or (iii) an enzyme to hydrolyze sialyl-residues and an enzyme to remove N-glycans from glycoprotein Asn-residues.

18. The kit according to claim 16, further comprising a TMT labelling reagent, and optionally a proteolytic digestion enzyme.

19. The method according to claim 1, wherein a peptide: N-glycosidase F (PNGAseF) is used to remove the N-glycans present on the glycoproteins.

20. The method according to claim 1, wherein the sialyltransferase-substrate is Cytosine 5′-monophosphate N-acetylneuraminic acid (CMP-Neu5Ac).
